(19) United States
(12) Patent Application Publication
(10) Pub. No.: US 2019/0083620 A1
(43) Pub. Date: Mar. 21, 2019

(54) METHODS FOR INDUCING AN IMMUNE RESPONSE AGAINST HUMAN IMMUNODEFICIENCY VIRUS INFECTION IN SUBJECTS UNDERGOING ANTIRETROVIRAL TREATMENT

(71) Applicants: Janssen Vaccines & Prevention B.V., Leiden (NL); Beth Israel Deaconess Medical Center, Inc., Boston, MA (US)

(72) Inventors: Maria Grazia PAU, Leiden (NL); Frank TOMAKA, Titusville, NJ (US); Daniel John STIEH, Oegstgeest (NL); Michal SARNECKI, Basel (CH); Dan BAROUCH, Newton, MA (US); Boris JUELG, Winchester, MA (US)

(21) Appl. No.: 16/132,555

(22) Filed: Sep. 17, 2018

Related U.S. Application Data
(60) Provisional application No. 62/559,881, filed on Sep. 18, 2017.

(51) Int. Cl. A61K 39/42; A61P 31/18; A61K 39/12; C12N 15/85

(52) U.S. Cl. CPC A61K 39/42; A61P 31/18; A61K 2039/51; C12N 15/85; A61K 39/12

(57) ABSTRACT

Methods for inducing an immune response against Human Immunodeficiency Virus (HIV) in HIV-infected subjects undergoing antiretroviral therapy (ART) are described. The methods include administering an adenovirus vector primer vaccine and either a Modified Vaccinia Ankara virus (MVA) vector booster vaccine or adenovirus booster vaccine in combination with isolated HIV envelope polypeptides.

### CROSS REFERENCE TO RELATED APPLICATIONS

This application is entitled to priority pursuant to 35 U.S.C. § 119(e) to U.S. Provisional Patent Application No. 62/559,881, filed Sep. 18, 2017, the disclosure of which is incorporated by reference herein in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “688097_373 Sequence Listing”, creation date of Sep. 18, 2017, and having a size of 73.7 KB. The sequence listing submitted via EFS-Web is part of the specification and is herein incorporated by reference in its entirety

### BACKGROUND OF THE INVENTION

The number of new HIV infections and the number of acquired immunodeficiency syndrome (AIDS) related deaths are declining. Nevertheless, globally, an estimated 36.7 million people were living with human immunodeficiency virus (HIV) in 2016 (http://www.unaids.org/en/resources/fact-sheet), which is an increase from previous years as a result of the wider availability of life-saving antiretroviral therapies (ART).

Despite its proven success at suppressing viral replication and saving lives, there are significant challenges to initiating and maintaining ART for all of those HIV-infected patients that need it in the world. For example, ART does not eliminate the viral reservoir, and treatment is associated with an incomplete restoration of the host immune system. In particular, while ART facilitates CD4⁺ T cell reconstruction in the blood, there is only a limited improvement in the function of anti-HIV specific CD8⁺ T cell responses. Also, ART must be taken life-long with near perfect adherence in order to be effective. This places extreme pressure and costs on international donors and over-taxed health systems in developing countries where HIV prevalence rates are highest. Moreover, ART has both short-term and long-term side effects for users, and drug resistance rates rise as more people are on treatment for longer periods of time. Thus, alternative or complementary treatments, including a therapeutic vaccine, which could induce a true or “functional” cure of HIV infection and lessen or eliminate the need for lifelong ART for HIV-infected individuals, would therefore be of great benefit. The concept of a “functional cure” includes therapeutic strategies that enable host control of the virus without the need for treatment.

Studies of HIV vaccine in HIV-uninfected and infected subjects suggest that a successful HIV vaccine program will need to induce immunity against the diverse strains and subtypes that are predominant in the target populations. Improving magnitude, breadth and depth of epitope coverage is thought to be a key to developing a successful T-cell based preventive HIV vaccine. Published primate data indicate that the number of epitope specific responses induced by a vaccine may be an important immune correlate of viral load control in the simian immunodeficiency virus (SIV) challenge system (Chen et al. Nat Med. (2001) 7(11), 1225-31). Strategies to accomplish this include using multivalent vaccines containing immunogens from a number of prevalent subtypes or using mosaic sequences, i.e., proteins assembled from natural sequences by in silico recombination, which are optimized for potential T-cell epitopes.

The enhancement of host-mediated clearance of residual virus represents a new additional approach to an HIV functional cure (Carcelain et al. Immunol Rev. (2013) 254(1), 355-71). Findings of several studies have shown the importance of cellular immunity in the control of HIV reservoir size. HIV-1 Gag-specific CD8⁺ T cells isolated from elite controllers, but not from patients given ART, were shown to kill autologous resting CD4+ T cells in which the virus was reactivated with vorinostat. Moreover, functional anti-viral CD8 T cells are associated with reduced size of the central memory CD4⁺ T cell reservoir in patients controlling their virus without ART. High-avidity multifunctional CD8⁺ cytotoxic T lymphocytes (CTL) that target vulnerable regions in Gag are especially important in limiting virus diversity and reservoirs in individuals infected with HIV who have protective human leukocyte antigen (HLA) class I alleles. Therapeutic vaccines could re-stimulate CD8⁺ CTL to prevent or control virus relapses and re-establish latent infection in CD4⁺ T cells after treatment interruptions. A few therapeutic vaccine studies, such as the Ad5 HIV-1 gag vaccine (ACTG A5197 NCT00080106), and infusions of dendritic cells pulsed with inactivated HIV particles have shown transient viral suppression after treatment interruption. Eramune-02 is testing whether a deoxyribonucleic acid (DNA) prime, replication defective, recombinant adenovirus serotype-5 boost strategy, with the Vaccine Research Center's polyvalent HIV-Gag, Pol, Nef, and Env vaccine can reduce the viral reservoir in patients undergoing an ARV-intensification regimen (Katlama Lancet. (2013) 381(9883), 2109-17).

Several studies indicated the immunogenicity of different vaccine approaches in HIV infected persons treated during chronic infection and that underwent analytic treatment interruption (ATI). For example, Garcia et al. and Tung et al. observed a decrease of plasma viral load associated with a significant increase in HIV-1 specific T cell response after vaccination and subsequent ART interruption (Garcia et al. Sci. Transl. Med. (2013) 5(166), 166ra2; Tung et al. Vaccine (2016) 34(19), 2225-32). In contrast, Pollard et al. showed that while a significant difference in viral load was noted between the vaccinated group and placebo group, the percentage changes in CD4⁺ T cell counts were not significant between the two groups (Pollard et al. Lancet Infect. Dis. (2014) 14(4), 291-300). Despite the fact that studied vaccines demonstrated significant anti-HIV-1 activity and, after ATI, many participants had rapid decline of viral load (after the peak rebound), none of them was able to maintain undetectable viral loads without ART.

In contrast to patients who initiated ART in the course of chronic HIV infection, many patients who begin ART at the time of acute HIV infection demonstrate blunted or delayed rebound viremia after analytical treatment interruption (ATI) (Gianella et al. Antiviral therapy. (2011) 16(4), 535-45; Goujard et al. (2012) Antiviral therapy. 17(6), 1001-9; Hamlyn et al. (2012) PloS one. 7(8), e43754; Lodi et al. (2012) Archives of internal medicine. 172(16), 1252-5; Saez-Cirion et al. (2013) PLoS pathogens 9(3), e1003211). Several studies have shown sustained viremic control after treatment interruption in 5%-16% of patients initiated on ART at the time of acute infection (Gianella 2011, supra; Goujard 2012, supra; Grijsen 2012, supra; Lodi 2012, supra; Saez-Cirion 2013, supra). In these studies, factors associated with successful viremic control included shorter duration from HIV onset to ART initiation, longer duration on ART and low PBMC-associated HIV DNA (Williams et al. (2014) Elife 3, e03821).

The possibility of safely stopping or interrupting ART would hold great benefit both for patients, who are inconvenienced by having to take medications that require strict adherence and have a number of proven short-term and long-term toxicities, and by national health programs, which are committed to providing medications to hundreds of thousands or even millions of patients for decades to come. Moreover, previous vaccine-based immunotherapy strategies failed to sufficiently control viral load in chronically HIV-infected subjects.

Accordingly, there is a need in the art for improved methods of treating HIV-infected subjects, particularly HIV-infected subjects undergoing antiretroviral therapy (ART) including chronically HIV-infected HIV subjects, such as therapeutic vaccines. Such a therapeutic vaccine preferably would improve immune responses to HIV and possibly allow at least some treated subjects to discontinue ART while maintaining viremic control.

### BRIEF SUMMARY OF THE INVENTION

The invention relates to methods for inducing an immune response against human immunodeficiency virus (HIV) in HIV-infected subjects undergoing antiretroviral therapy (ART) with a primer vaccine of adenovirus 26 (Ad26) vectors encoding mosaic HIV antigens and either a booster vaccine of Modified Vaccinia Ankara (MVA) vectors encoding mosaic HIV antigens or a booster vaccine of Ad26 vectors encoding mosaic HIV antigens, alone or in combination with isolated gp140 proteins.

In one general aspect, the invention relates to a method of inducing an immune response against a human immunodeficiency virus (HIV) in an HIV-infected human subject undergoing antiretroviral therapy (ART), the method comprising administering to the human subject:
(i) a primer vaccine comprising an immunogenically effective amount of one or more adenovirus 26 (Ad26) vectors together encoding four HIV antigens having the amino acid sequences of SEQ ID NO: 1, SEQ ID NO: 12, SEQ ID NO: 3, and SEQ ID NO: 4, and a pharmaceutically acceptable carrier; and
(ii) a booster vaccine comprising an immunogenically effective amount of one or more Modified Vaccinia Ankara (MVA) vectors together encoding four HIV antigens having the amino acid sequences of SEQ ID NO: 1, SEQ ID NO: 3, SEQ ID NO: 4, and either one of SEQ ID NO: 2 or SEQ ID NO: 12, and a pharmaceutically acceptable carrier.

In certain embodiments, the immunogenically effective amount of the one or more Ad26 vectors encoding the four HIV antigens consists of a first Ad26 vector encoding the HIV antigen of SEQ ID NO: 1, a second Ad26 vector encoding the HIV antigen of SEQ ID NO: 12, a third Ad26 vector encoding the HIV antigen of SEQ ID NO: 3, and a fourth Ad26 vector encoding the HIV antigen of SEQ ID NO: 4.

In some embodiments, the immunogenically effective amount of the one or more MVA vectors encoding the four HIV antigens consists of a single MVA vector encoding the HIV antigens of SEQ ID NOs: 1, 3, 4, and either one of SEQ ID NOs: 2 or 12.

In other embodiments, the immunogenically effective amount of the one or more MVA vectors encoding the four HIV antigens consists of a first MVA vector encoding the HIV antigens of SEQ ID NOs: 1 and 3, and a second MVA vector encoding the HIV antigen of SEQ ID NO: 4 and either one of the HIV antigens of SEQ ID NOs: 2 or 12.

In certain embodiments, the first, second, third, and fourth Ad26 vectors together are administered at a total dose of about 5×10⁹ to about 1×10¹¹ viral particles (vp), preferably about 5×10¹⁰ vp, of the Ad26 vectors; and the single MVA vector or the first and second MVA vectors together are administered at a total dose of about 1×10⁷ to about 5×10⁸ plaque-forming units (pfu), preferably about 1×10⁸ pfu, of the MVA vector or vectors.

In some embodiments, the method further comprises administering to the human subject one or more isolated HIV gp140 envelope polypeptides in combination with the booster vaccine, preferably one or more isolated HIV gp140 envelope polypeptides selected from the group consisting of two trimeric HIV gp140 envelope polypeptides having the amino acid sequences of SEQ ID NO: 9 and SEQ ID NO: 10.

In another general aspect, the invention relates to a method of inducing an immune response against a human immunodeficiency virus (HIV) in an HIV-infected human subject undergoing antiretroviral therapy (ART), the method comprising administering to the human subject:
(i) a primer vaccine comprising an immunogenically effective amount of one or more adenovirus 26 (Ad26) vectors together encoding four HIV antigens having the amino acid sequences of SEQ ID NO: 1, SEQ ID NO: 12, SEQ ID NO: 3, and SEQ ID NO: 4, and a pharmaceutically acceptable carrier, in a total dose of about 5×10⁹ to about 1×10¹¹ viral particles (vp), preferably about 5×10¹⁰ vp, of the Ad26 vectors; and
(ii) a booster vaccine comprising:
   (ii,a) a first booster vaccine composition comprising an immunogenically effective amount of one or more adenovirus 26 (Ad26) vectors together encoding four HIV antigens having the amino acid sequences of SEQ ID NO: 1, SEQ ID NO: 12, SEQ ID NO: 3, and SEQ ID NO: 4, and a pharmaceutically acceptable carrier, in a total dose of about 5×10⁹ to about 1×10¹¹ viral particles (vp), preferably about 5×10¹⁰ vp, of the Ad26 vectors; and
   (ii,b) a second booster vaccine composition comprising at least one isolated HIV gp140 envelope polypeptide having an amino acid sequence selected from the group consisting of SEQ ID NO: 9 and SEQ ID NO: 10, an aluminum phosphate adjuvant and a pharmaceutically acceptable carrier, at a total dose of about 125 μg to 350 μg, preferably about 250 μg, of the at least one isolated HIV gp140 envelope polypeptide,
      wherein the first and second booster vaccine compositions are administered in combination.

In particular embodiments, the second booster vaccine composition comprises two trimeric HIV gp140 envelope polypeptides having the amino acid sequences of SEQ ID NO: 9 and SEQ ID NO: 10.

In certain embodiments of the methods of the invention, the booster vaccine is first administered at about 12-26 weeks, e.g., about 24 weeks, after the primer vaccine is initially administered.

In one embodiment of the methods of the invention, the primer vaccine is re-administered at about 10-14 weeks, e.g., 12 weeks, after the primer vaccine is initially administered; and the booster vaccine is re-administered at about 34-38 weeks, e.g., 36 weeks, after the primer vaccine is initially administered.

In some embodiments, the subject is a chronically HIV-infected subject. In such embodiments, the subject preferably initiated ART outside of the acute phase of HIV infection.

In other embodiments of the invention, the method comprises further administering to the human subject a toll-like receptor 7 (TLR7) agonist.

The invention also relates to a vaccine combination for use in inducing an immune response against a human immunodeficiency virus (HIV) in an HIV-infected subject undergoing antiretroviral therapy (ART); and use of a vaccine combination in the manufacture of a medicament for inducing an immune response against a human immunodeficiency virus (HIV) in an HIV-infected subject undergoing antiretroviral therapy (HIV).

In one embodiment, the vaccine combination comprises: (i) a primer vaccine comprising an immunogenically effective amount of one or more adenovirus 26 (Ad26) vectors together encoding four HIV antigens having the amino acid sequences of SEQ ID NO:1, SEQ ID NO: 12, SEQ ID NO: 3, and SEQ ID NO: 4, and a pharmaceutically acceptable carrier; and (ii) a booster vaccine comprising an immunogenically effective amount of one or more Modified Vaccinia Ankara (MVA) vectors together encoding four HIV antigens having the amino acid sequences of SEQ ID NO: 1, SEQ ID NO: 3, SEQ ID NO: 4, and either one of SEQ ID NO: 2 or SEQ ID NO: 12; and a pharmaceutically acceptable carrier.

In another embodiment, the vaccine combination comprises (i) a primer vaccine comprising an immunogenically effective amount of one or more adenovirus 26 (Ad26) vectors together encoding four HIV antigens having the amino acid sequences of SEQ ID NO:1, SEQ ID NO: 12, SEQ ID NO: 3, and SEQ ID NO: 4, and a pharmaceutically acceptable carrier; and (ii) a booster vaccine comprising: (ii, a) a first booster vaccine composition comprising an immunogenically effective amount of one or more adenovirus 26 (Ad26) vectors together encoding four HIV antigens having the amino acid sequences of SEQ ID NO: 1, SEQ ID NO: 12, SEQ ID NO: 3, and SEQ ID NO: 4, and a pharmaceutically acceptable carrier; and (ii, b) a second booster vaccine composition comprising at least one isolated HIV gp140 envelope polypeptide having an amino acid sequence selected from the group consisting of SEQ ID NO: 9 and SEQ ID NO: 10, an aluminum phosphate adjuvant and a pharmaceutically acceptable carrier.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, “subject” means a human, who will be or has been treated by a method according to an embodiment of the invention.

The terms “adjuvant” and “immune stimulant” are used interchangeably herein, and are defined as one or more substances that cause stimulation of the immune system. In this context, an adjuvant is used to enhance an immune response to HIV antigens and antigenic HIV polypeptides of the invention.

As used herein, the terms and phrases “in combination,” “in combination with,” “co-delivery,” and “administered together with” in the context of the administration of two or more therapies or components to a subject refers to simultaneous administration of two or more therapies or components, such as a viral expression vector and an isolated antigenic polypeptide. “Simultaneous administration” can be administration of the two components at least within the same day. When two components are “administered together with” or “administered in combination with,” they can be administered in separate compositions sequentially within a short time period, such as 24, 20, 16, 12, 8 or 4 hours, or within 1 hour, or they can be administered in a single composition at the same time. In the typical embodiment, two components or therapies are administered in separate compositions. The use of the term “in combination with” does not restrict the order in which therapies or components are administered to a subject. For example, a first therapy or component (e.g. viral expression vector) can be administered prior to (e.g., 5 minutes to one hour before), concomitantly with or simultaneously with, or subsequent to (e.g., 5 minutes to one hour after) the administration of a second therapy (e.g., isolated HIV antigenic polypeptide).

The invention relates to methods of priming and boosting an immune response against human immunodeficiency virus (HIV) in an HIV-infected human subject undergoing antiretroviral treatment (ART). According to embodiments of the invention, a primer vaccine comprises an immunogenically effective amount of one or more adenovirus 26 (Ad26) vectors encoding one or more mosaic HIV antigens. In some embodiments of the invention, a booster vaccine comprises an immunogenically effective amount of one or more Modified Vaccinia Ankara (MVA) vectors encoding one or more mosaic HIV antigens. In other embodiments of the invention, a booster vaccine comprises a first booster vaccine composition comprising an immunogenically effective amount of one or more adenovirus 26 (Ad26) vectors encoding one or more mosaic HIV antigens; and a second booster vaccine composition comprising one or more isolated HIV gp140 proteins.

Human Immunodeficiency Virus (HIV)

Human immunodeficiency virus (HIV) is a member of the genus Lentivirinae, which is part of the family of Retroviridae. Two species of HIV infect humans: HIV-1 and HIV-2. HIV-1 is the most common strain of HIV virus, and is known to be more pathogenic than HIV-2. As used herein, the terms “human immunodeficiency virus” and “HIV” refer, but are not limited to, HIV-1 and HIV-2. In preferred embodiments, the envelope proteins described herein refer to those present on HIV-1.

HIV is categorized into multiple clades with a high degree of genetic divergence. As used herein, the term “HIV clade” or “HIV subtype” refers to related human immunodeficiency viruses classified according to their degree of genetic similarity. There are currently three groups of HIV-1 isolates: M, N and O. Group M (major strains) consists of at least ten clades, A through J. Group O (outer strains) can consist of a similar number of clades. Group N is a new HIV-1 isolate that has not been categorized in either group M or O.

According to embodiments of the invention, the methods described herein can be used to induce an immune response against one or more clades of HIV.

HIV Antigens

As used herein, the terms “HIV antigen,” “antigenic polypeptide of an HIV,” “HIV antigenic polypeptide,” “HIV antigenic protein,” “HIV immunogenic polypeptide,” and “HIV immunogen” all refer to a polypeptide capable of inducing an immune response, e.g., a humoral and/or cellular mediated response, against HIV in a subject. The HIV antigen can be a protein of HIV, a fragment or epitope thereof, or a combination of multiple HIV proteins or portions thereof, that can induce an immune response against HIV in a subject. An HIV antigen is capable of raising in a host a protective immune response, e.g., inducing an immune response against a viral disease or infection, and/or producing an immunity in (i.e., vaccinates) a subject against a viral disease or infection, that protects the subject against the viral disease or infection. For example, the HIV antigen can comprise a protein or fragment(s) thereof from HIV, such as the HIV gag, pol and env gene products.

According to embodiments of the invention, the HIV antigen can be an HIV-1 or HIV-2 antigen or fragment(s) thereof. Examples of HIV antigens include, but are not limited to gag, pol, and env gene products, which encode structural proteins and essential enzymes. Gag, pol, and env gene products are synthesized as polyproteins, which are further processed into multiple other protein products. The primary protein product of the gag gene is the viral structural protein gag polyprotein, which is further processed into MA, CA, SP1, NC, SP2, and P6 protein products. The pol gene encodes viral enzymes (Pol, polymerase), and the primary protein product is further processed into RT, RNase H, IN, and PR protein products. The env gene encodes structural proteins, specifically glycoproteins of the virion envelope. The primary protein product of the env gene is gp160, which is further processed into gp120 and gp41. A heterologous nucleic acid sequence according to the invention preferably encodes a gag, env, and/or pol gene product, or portion thereof. According to a preferred embodiment, the HIV antigen comprises an HIV Gag, Env, or Pol antigen, or any portion or combination thereof, more preferably an HIV-1 Gag, Env, or Pol antigen, or any portion or combination thereof

According to preferred embodiments of the invention, an HIV antigen is a mosaic HIV antigen. As used herein, “mosaic antigen” refers to a recombinant protein assembled from fragments of natural sequences. The “mosaic antigen” can be computationally generated and optimized using a genetic algorithm. Mosaic antigens resemble natural antigens, but are optimized to maximize the coverage of potential T-cell epitopes found in the natural sequences, which improves the breadth and coverage of the immune response.

Examples of mosaic HIV Gag-Pol-Env antigens include those described in, e.g., US20120076812; Barouch et al., Nat Med 2010, 16:319-323; Barouch et al., Cell 155:1-9, 2013; and WO 2017/102929, all of which are incorporated herein by reference in their entirety.

Preferably, the mosaic HIV antigens encoded by the vectors according to the invention comprise one or more of the amino acid sequences selected from the group consisting of SEQ ID NOs: 1-4 and 12. Alternative and/or additional HIV antigens can be encoded by the primer vaccine and/or the booster vaccine of the invention in certain embodiments, e.g. to further broaden the immune response.

In view of the present disclosure, a mosaic HIV antigen can be produced using methods known in the art. See, e.g., US20120076812; Fischer et al, Nat Med, 2007. 13(1): p. 100-6; Barouch et al., Nat Med 2010, 16:319-323, all of which are incorporated herein by reference in their entirety.

Envelope Polypeptide

As used herein, each of the terms “envelope polypeptide,” “envelope glycoprotein,” “env polypeptide,” “env glycoprotein,” and “Env” refers to, but is not limited to, the glycoprotein that is expressed on the surface of the envelope of HIV virions and the surface of the plasma membrane of HIV infected cells, or a fragment thereof that can induce an immune response or produce an immunity against HIV in a subject in need thereof.

The env gene encodes gp160, which is proteolytically cleaved into gp120 and gp41. More specifically, gp160 trimerizes to (gp160)₃ and then undergoes cleavage into the two noncovalently associated fragments gp120 and gp41. Viral entry is subsequently mediated by a trimer of gp120/gp41 heterodimers. Gp120 is the receptor binding fragment, and binds to the CD4 receptor on a target cell that has such a receptor, such as, e.g., a T-helper cell. Gp41, which is non-covalently bound to gp120, is the fusion fragment and provides the second step by which HIV enters the cell. Gp41 is originally buried within the viral envelope, but when gp120 binds to a CD4 receptor, gp120 changes its conformation causing gp41 to become exposed, where it can assist in fusion with the host cell. Gp140 is the uncleaved ectodomain of trimeric gp160, i.e., (gp160)₃, that has been used as a surrogate for the native state of the cleaved, viral spike.

According to one embodiment of the invention, isolated HIV envelope polypeptides (e.g., gp160, gp140, gp120, or gp41), preferably gp140 protein, and more preferably stabilized trimeric gp140 protein, can be administered for priming or boosting immunizations, preferably boosting immunizations, to enhance the immunity induced by expression vectors (e.g., adenovirus 26 and/or MVA vectors) alone.

As used herein, each of the terms “stabilized trimeric gp140 protein” and “stabilized trimer of gp140” refers to a trimer of gp140 polypeptides that includes a polypeptide sequence that increases the stability of the trimeric structure. The gp140 polypeptides can have, or can be modified to include a trimerization domain that stabilizes trimers of gp140. Examples of trimerization domains include, but are not limited to, the T4-fibritin “foldon” trimerization domain; the coiled-coil trimerization domain derived from GCN4; and the catalytic subunit of E. coli aspartate transcarbamoylase as a trimer tag.

Examples of isolated antigenic polypeptide are stabilized trimeric gp140 such as those described in Nkolola et al 2010, J. Virology 84(7): 3270-3279; Kovacs et al, PNAS 2012, 109(30):12111-6; WO 2010/042942 and WO 2014/107744, all of which are incorporated by reference in their entirety.

In some embodiments of the invention, the “envelope polypeptide” or “envelope glycoprotein” is a mosaic envelope protein comprising multiple epitopes derived from one or more of Env polyprotein sequences of one or more HIV clades. For example, as used herein a “gp140 protein” can be a “mosaic gp140 protein” that contains multiple epitopes derived from one or more gp140 protein sequences of one or more HIV clades. Preferably, a mosaic gp140 protein is a stabilized trimeric gp140 protein.

In a preferred embodiment, a mosaic gp140 protein is a stabilized trimer of mosaic gp140 protein comprising the amino acid sequence of SEQ ID NO: 10.

In some embodiments of the invention, the envelope polypeptide” or “envelope glycoprotein” is an envelope protein derived from a particular HIV clade, such as HIV clade A, B, or C. For example, as used herein a “gp140 protein” can be a “clade C gp140 protein” that contains envelope protein sequence derived from HIV clade C. Preferably, a clade C gp140 protein is a stabilized trimeric clade C gp140 protein.

In a preferred embodiment, a clade C gp140 protein is a stabilized trimer of clade C gp140 protein comprising the amino acid sequence of SEQ ID NO: 9.

According to one embodiment of the invention, a gp140 polypeptide, such as a stabilized trimeric gp140 protein can be administered as a boosting immunization or as a component of a boosting immunization together with viral expression vectors, e.g., adenovirus 26 and/or MVA vectors.

In certain embodiments of the invention, two gp140 proteins are administered to the same subject, preferably a clade C gp140 having the amino acid sequence of SEQ ID NO: 9 and a mosaic gp140 having the amino acid sequence of SEQ ID NO: 10. The two gp140 proteins can be together in one pharmaceutical composition, preferably administered together with an adjuvant, such as aluminum phosphate adjuvant. A preferred dose for the total amount of gp140 for administration to humans is between about 125 and 350 μg, preferably about 250 μg. If clade C gp140 and mosaic gp140 are both administered, a suitable dose would for instance be about 125 μg of each protein, to provide a total dose of 250 μg of gp140 protein for an administration to a human subject.

An isolated gp140 protein can be co-delivered or administered in combination with an adenovirus (e.g., Ad26) expression vector or MVA expression vector. According to a preferred embodiment, a gp140 protein and Ad26 or MVA vector are administered separately, as two distinct formulations. Alternatively, a gp140 protein can be administered with Ad26 or MVA together in a single formulation. Simultaneous administration or co-delivery can take place at the same time, within one hour, or within the same day. Furthermore, a gp140 protein can be administered in an adjuvanted formulation. Suitable adjuvants can be, for example, aluminum phosphate or a saponin-based adjuvant, preferably aluminum phosphate adjuvant.

Antigenic polypeptides can be produced and isolated using any method known in the art in view of the present disclosure. For example, an antigenic polypeptide can be expressed from a host cell, preferably a recombinant host cell optimized for production of the antigenic polypeptide. According to an embodiment of the invention, a recombinant gene is used to express a gp140 protein containing mutations to eliminate cleavage and fusion activity, preferably an optimized gp140 protein with increased breadth, intensity, depth, or longevity of the antiviral immune response (e.g., cellular or humoral immune responses) generated upon immunization (e.g., when incorporated into a composition of the invention, e.g., vaccine of the invention) of a subject (e.g., a human). The optimized gp140 protein can also include cleavage site mutation(s), a factor Xa site, and/or a foldon trimerization domain. A leader/signal sequence can be operably linked to the N-terminus of an optimized gp140 protein for maximal protein expression. The leader/signal sequence is usually cleaved from the nascent polypeptide during transport into the lumen of the endoplasmic reticulum. Any leader/signal sequence suitable for a host cell of interest can be used. An exemplary leader/signal sequence comprises the amino acid sequence of SEQ ID NO: 11.

Adenovirus Vector

Primer vaccines, and in certain embodiments booster vaccines, used in the methods of the invention comprise one or more adenovirus vectors, particularly human adenovirus 26 vectors (Ad26) encoding one more mosaic HIV antigens. An adenovirus according to the invention belongs to the family of the Adenoviridae, and preferably is one that belongs to the genus Mastadenovirus. As used herein, the notation “rAd” means recombinant adenovirus, e.g., “rAd26” refers to recombinant human adenovirus 26.

According to embodiments of the invention, an adenovirus is a human adenovirus serotype 26 (Ad26). An advantage of human adenovirus serotype 26 is a low seroprevalence and/or low pre-existing neutralizing antibody titers in the human population. In some embodiments, the adenovirus vector is a replication deficient recombinant viral vector, such as a replication deficient recombinant adenovirus 26 vector.

An “adenovirus capsid protein” refers to a protein on the capsid of an adenovirus (e.g., Ad26 vectors) that is involved in determining the serotype and/or tropism of a particular adenovirus. Adenoviral capsid proteins typically include the fiber, penton and/or hexon proteins. An rAd26 vector comprises at least the hexon of Ad26, preferably at least the hexon and fiber of Ad26. In preferred embodiments, the hexon, penton and fiber are of Ad26. Preferably, also the non-capsid proteins are from Ad26.

In certain embodiments, the recombinant adenovirus vector useful in the invention is derived mainly or entirely from Ad26 (i.e., the vector is rAd26). In some embodiments, the adenovirus is replication deficient, e.g., because it contains a deletion in the E1 region of the genome. For the adenoviruses derived from Ad26 used in the invention, it is typical to exchange the E4-orf6 coding sequence of the adenovirus with the E4-orf6 of an adenovirus of human subgroup C such as Ad5. This allows propagation of such adenoviruses in well-known complementing cell lines that express the E1 genes of AdS, such as for example 293 cells, PER.C6 cells, and the like (see, e.g. Havenga, et al., 2006, J Gen Virol 87: 2135-43; WO 03/104467). However, such adenoviruses will not be capable of replicating in non-complementing cells that do not express the E1 genes of AdS. Thus, in certain embodiments, the adenovirus is a human adenovirus of serotype 26, with a deletion in the E1 region into which the nucleic acid encoding one or more mosaic HIV antigens has been cloned, and with an E4 orf6 region of AdS.

The preparation of recombinant adenoviral vectors is well known in the art. Preparation of rAd26 vectors is described, for example, in WO 2007/104792 and in Abbink et al., (2007) Virol 81(9): 4654-63, both of which are incorporated by reference herein in their entirety. Exemplary genome sequences of Ad26 are found in GenBank Accession EF 153474 and in SEQ ID NO: 1 of WO 2007/104792, which is herein incorporated by reference in its entirety. Typically, an adenovirus vector useful in the invention is produced using a nucleic acid comprising the entire recombinant adenoviral genome (e.g., a plasmid, cosmid, or baculovirus vector).

The adenovirus vectors useful in the invention are typically replication deficient. In these embodiments, the virus is rendered replication deficient by deletion or inactivation of regions critical to replication of the virus, such as the E1 region. The regions can be substantially deleted or inactivated by, for example, inserting a gene of interest, such as a gene encoding an HIV antigen (usually linked to a promoter) within the region. In some embodiments, the vectors of the invention can contain deletions in other regions, such as the E3 region, or insertions of heterologous genes linked to a promoter within such regions. Mutations in the E3 region of the adenovirus need not be complemented by the cell line, since E3 is not required for replication.

A packaging cell line is typically used to produce sufficient amounts of adenovirus vectors for use in the invention. A packaging cell is a cell that comprises those genes that have been deleted or inactivated in a replication deficient vector, thus allowing the virus to replicate in the cell. Suitable packaging cell lines include, for example, PER.C6, 911, and HEK293.

According to embodiments of the invention, any mosaic HIV antigen can be expressed in the adenovirus 26 vectors described herein. Optionally, the heterologous gene encoding the mosaic HIV antigen can be codon-optimized to ensure proper expression in the treated host (e.g., human). Codon-optimization is a technology widely applied in the art. Typically, the heterologous gene encoding the mosaic HIV antigen is cloned into the E1 and/or the E3 region of the adenoviral genome. Non-limiting embodiments of codon optimized nucleotide sequences encoding HIV antigens with SEQ ID NOs: 1-4 and 8 are provided herein as SEQ ID NOs: 5-8 and 13, respectively.

In a preferred embodiment of the invention, one or more adenovirus 26 (Ad26) vectors comprise nucleic acid that encodes one or more mosaic HIV antigens. In other preferred embodiments, the one or more Ad26 vectors encode one or more HIV antigens comprising the amino acid sequences selected from the group consisting of SEQ ID NOs: 1-4 and 12, and more preferably together encode four mosaic HIV antigens having the amino acid sequences of SEQ ID NO: 1, SEQ ID NO: 12, SEQ ID NO: 3, and SEQ ID NO: 4.

The heterologous gene encoding the mosaic HIV antigen can be under the control of (i.e., operably linked to) an adenovirus-derived promoter (e.g., the Major Late Promoter), or can be under the control of a heterologous promoter. Examples of suitable heterologous promoters include the cytomegalovirus (CMV) promoter and the Rous sarcoma virus (RSV) promoter. Preferably, the promoter is located upstream of the heterologous gene encoding the mosaic HIV antigen within an expression cassette. In a preferred embodiment, the heterologous promoter is a CMV promoter.

In a preferred embodiment of the invention, the adenovirus vectors are rAd26 vectors, such as that described in Abbink, J Virol, 2007. 81(9): p. 4654-63, which is incorporated herein by reference.

MVA Vectors

In some embodiments, booster vaccines used in the methods of the invention comprise one or more Modified Vaccinia Ankara (MVA) vectors encoding one more mosaic HIV antigens. MVA vectors useful in the invention utilize attenuated virus derived from MVA virus, which is characterized by the loss of their capabilities to reproductively replicate in human cell lines. The MVA vectors can express any HIV antigens known to those skilled in the art, preferably mosaic HIV antigens, including but not limited to the mosaic HIV antigens discussed herein.

MVA has been generated by more than 570 serial passages on chicken embryo fibroblasts of the dermal vaccinia strain Ankara (Chorioallantois vaccinia virus Ankara virus, CVA; for review see Mayr et al. (1975) Infection 3, 6-14) that was maintained in the Vaccination Institute, Ankara, Turkey for many years and used as the basis for vaccination of humans. However, due to the often severe post-vaccination complications associated with vaccinia viruses, there were several attempts to generate a more attenuated, safer smallpox vaccine.

During the period of 1960 to 1974, Prof. Anton Mayr succeeded in attenuating CVA by over 570 continuous passages in CEF cells (Mayr et al. (1975), Infection 3, 6-14). It was shown in a variety of animal models that the resulting MVA was avirulent (Mayr, A. & Danner, K. (1978), Dev. Biol. Stand. 41: 225-234). As part of the early development of MVA as a pre-smallpox vaccine, there were clinical trials using MVA-517 in combination with Lister Elstree (Stickl (1974), Prev. Med. 3: 97-101; Stickl and Hochstein-Mintzel (1971), Munch. Med. Wochenschr. 113: 1149-1153) in subjects at risk for adverse reactions from vaccinia. In 1976, MVA derived from MVA-571 seed stock (corresponding to the 571st passage) was registered in Germany as the primer vaccine in a two-stage parenteral smallpox vaccination program. Subsequently, MVA-572 was used in approximately 120,000 Caucasian individuals, the majority being children between 1 and 3 years of age, with no reported severe side effects, even though many of the subjects were among the population with high risk of complications associated with vaccinia (Mayr et al. (1978), Zentralbl. Bacteriol. (B) 167:375-390). MVA-572 was deposited at the European Collection of Animal Cell Cultures as ECACC V94012707.

As a result of the passaging used to attenuate MVA, there are a number of different strains or isolates, depending on the number of passages conducted in CEF cells. For example, MVA-572 was used in a small dose as a pre-vaccine in Germany during the smallpox eradication program, and MVA-575 was extensively used as a veterinary vaccine. MVA as well as MVA-BN lacks approximately 15% (31 kb from six regions) of the genome compared with ancestral CVA virus. The deletions affect a number of virulence and host range genes, as well as the gene for Type A inclusion bodies. MVA-575 was deposited on Dec. 7, 2000, at the European Collection of Animal Cell Cultures (ECACC) under Accession No. V00120707. The attenuated CVA-virus MVA (Modified Vaccinia Virus Ankara) was obtained by serial propagation (more than 570 passages) of the CVA on primary chicken embryo fibroblasts.

Even though Mayr et al. demonstrated during the 1970s that MVA is highly attenuated and avirulent in humans and mammals, certain investigators have reported that MVA is not fully attenuated in mammalian and human cell lines, since residual replication might occur in these cells (Blanchard et al. (1998), J. Gen. Virol. 79:1159-116779; Carroll & Moss (1997), Virology 238:198-211; U.S. Pat. No. 5,185,146; 81). It is assumed that the results reported in these publications have been obtained with various known strains of MVA, since the viruses used essentially differ in their properties, particularly in their growth behavior in various cell lines. Such residual replication is undesirable for various reasons, including safety concerns in connection with use in humans.

Strains of MVA having enhanced safety profiles for the development of safer products, such as vaccines or pharmaceuticals, have been developed, for example by Bavarian Nordic. MVA was further passaged by Bavarian Nordic and is designated MVA-BN. A representative sample of MVA-BN was deposited on Aug. 30, 2000 at the European Collection of Cell Cultures (ECACC) under Accession No. V00083008. MVA-BN is further described in WO 02/42480 (US 2003/0206926) and WO 03/048184 (US 2006/0159699), both of which are incorporated by reference herein in their entirety.

“Derivatives” or “variants” of MVA refer to viruses exhibiting essentially the same replication characteristics as MVA as described herein, but exhibiting differences in one or more parts of their genomes. For example, MVA-BN as well as a derivative or variant of MVA-BN fails to reproductively replicate in vivo in humans and mice, even in severely immune suppressed mice. More specifically, MVA-BN or a derivative or variant of MVA-BN also preferably has the capability of reproductive replication in chicken embryo fibroblasts (CEF), but no capability of reproductive replication in the human keratinocyte cell line HaCat (Boukamp et al (1988), J. Cell Biol. 106: 761-771), the human bone osteosarcoma cell line 143B (ECACC Deposit No. 91112502), the human embryo kidney cell line 293 (ECACC Deposit No. 85120602), and the human cervix adenocarcinoma cell line HeLa (ATCC Deposit No. CCL-2). Additionally, a derivative or variant of MVA-BN has a virus amplification ratio at least two fold less, more preferably three-fold less than MVA-575 in Hela cells and HaCaT cell lines. Tests and assays for these properties of MVA variants are described in WO 02/42480 (US 2003/0206926) and WO 03/048184 (US 2006/0159699).

The term “not capable of reproductive replication” or “no capability of reproductive replication” is, for example, described in WO 02/42480, which also teaches how to obtain MVA having the desired properties as mentioned above. The term applies to a virus that has a virus amplification ratio at 4 days after infection of less than 1 using the assays described in WO 02/42480 or in U.S. Pat. No. 6,761,893, both of which are incorporated by reference herein in their entirety.

The term “fails to reproductively replicate” refers to a virus that has a virus amplification ratio at 4 days after infection of less than 1. Assays described in WO 02/42480 or in U.S. Pat. No. 6,761,893 are applicable for the determination of the virus amplification ratio.

The amplification or replication of a virus is normally expressed as the ratio of virus produced from an infected cell (output) to the amount originally used to infect the cell in the first place (input), and is referred to as the “amplification ratio.” An amplification ratio of “1” defines an amplification status where the amount of virus produced from the infected cells is the same as the amount initially used to infect the cells, meaning that the infected cells are permissive for virus infection and reproduction. In contrast, an amplification ratio of less than 1, i.e., a decrease in output compared to the input level, indicates a lack of reproductive replication and therefore attenuation of the virus.

The advantages of MVA-based vaccine include their safety profile as well as availability for large scale vaccine production. Furthermore, in addition to its efficacy, the feasibility of industrial scale manufacturing can be beneficial. Additionally, MVA-based vaccines can deliver multiple heterologous antigens and allow for simultaneous induction of humoral and cellular immunity.

MVA vectors useful for the invention can be prepared using methods known in the art, such as those described in WO/2002/042480, WO/2002/24224, US20110159036, U.S. Pat. No. 8,197,825, etc., the relevant disclosures of which are incorporated herein by reference.

In another aspect, replication deficient MVA viral strains can also be suitable for use in the invention, such as strains MVA-572 and MVA-575, or any other similarly attenuated MVA strain. Also suitable can be a mutant MVA, such as the deleted chorioallantois vaccinia virus Ankara (dCVA). A dCVA comprises del I, del II, del III, del IV, del V, and del VI deletion sites of the MVA genome. The sites are particularly useful for the insertion of multiple heterologous sequences. The dCVA can reproductively replicate (with an amplification ratio of greater than 10) in a human cell line (such as human 293, 143B, and MRC-5 cell lines), which then enables optimization by further mutation useful for a virus-based vaccination strategy (see, e.g., WO 2011/092029).

According to embodiments of the invention, the MVA vector(s) comprise a nucleic acid that encodes one or more HIV antigens having the amino acid sequences selected from the group consisting of SEQ ID NOs: 1-4 and 12. Preferably, the one or more MVA vectors together encode four mosaic HIV antigens having the amino acid sequences of SEQ ID NO: 1, SEQ ID NO: 3, SEQ ID NO: 4, and either one of SEQ ID NO: 2 or SEQ ID NO: 12.

Nucleic acid sequences encoding the mosaic HIV antigens can be inserted into one or more intergenic regions (IGR) of the MVA. In certain embodiments, the IGR is selected from IGR07/08, IGR 44/45, IGR 64/65, IGR 88/89, IGR 136/137, and IGR 148/149. In certain embodiments, less than 5, 4, 3, or 2 IGRs of the recombinant MVA comprise heterologous nucleotide sequences encoding an HIV antigen, such as a mosaic HIV antigen. The heterologous nucleotide sequences can, additionally or alternatively, be inserted into one or more of the naturally occurring deletion sites, in particular into the main deletion sites I, II, III, IV, V, or VI of the MVA genome. In certain embodiments, less than 5, 4, 3, or 2 of the naturally occurring deletion sites of the recombinant MVA comprise heterologous nucleotide sequences encoding mosaic HIV antigens.

The number of insertion sites of MVA comprising heterologous nucleotide sequences encoding HIV antigens can be 1, 2, 3, 4, 5, or more. In certain embodiments, the heterologous nucleotide sequences are inserted into 4, 3, 2, or fewer insertion sites. Preferably, two insertion sites are used. In certain embodiments, three insertion sites are used. Preferably, the recombinant MVA comprises at least 2, 3, 4, 5, 6, or 7 genes inserted into 2 or 3 insertion sites.

The recombinant MVA viruses provided herein can be generated by routine methods known in the art. Methods to obtain recombinant poxviruses or to insert exogenous coding sequences into a poxviral genome are well known to the person skilled in the art. For example, methods for standard molecular biology techniques such as cloning of DNA, DNA and RNA isolation, Western blot analysis, RT-PCR and PCR amplification techniques are described in Molecular Cloning, A laboratory Manual (2nd Ed.) (J. Sambrook et al., Cold Spring Harbor Laboratory Press (1989)), and techniques for the handling and manipulation of viruses are described in Virology Methods Manual (B. W. J. Mahy et al. (eds.), Academic Press (1996)). Similarly, techniques and know-how for the handling, manipulation and genetic engineering of MVA are described in Molecular Virology: A Practical Approach (A. J. Davison & R. M. Elliott (Eds.), The Practical Approach Series, IRL Press at Oxford University Press, Oxford, UK (1993) (see, e.g., Chapter 9: Expression of genes by Vaccinia virus vectors)) and Current Protocols in Molecular Biology (John Wiley & Son, Inc. (1998) (see, e.g., Chapter 16, Section IV: Expression of proteins in mammalian cells using vaccinia viral vector)).

For the generation of the various recombinant MVAs disclosed herein, different methods can be applicable. The DNA sequence to be inserted into the virus can be placed into an E. coli plasmid construct into which DNA homologous to a section of DNA of the MVA has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of MVA DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA. Recombination between homologous MVA DNA in the plasmid and the viral genome, respectively, can generate an MVA modified by the presence of foreign DNA sequences.

According to a preferred embodiment, a cell of a suitable cell culture such as, e.g., CEF cells, can be infected with a poxvirus. The infected cell can be, subsequently, transfected with a first plasmid vector comprising a foreign or heterologous gene or genes, preferably under the transcriptional control of a poxvirus expression control element. As explained above, the plasmid vector also comprises sequences capable of directing the insertion of the exogenous sequence into a selected part of the poxviral genome. Optionally, the plasmid vector also contains a cassette comprising a marker and/or selection gene operably linked to a poxviral promoter.

Suitable marker or selection genes are, e.g., the genes encoding the green fluorescent protein, β-galactosidase, neomycin-phosphoribosyltransferase or other markers. The use of selection or marker cassettes simplifies the identification and isolation of the generated recombinant poxvirus. However, a recombinant poxvirus can also be identified by PCR technology. Subsequently, a further cell can be infected with the recombinant poxvirus obtained as described above and transfected with a second vector comprising a second foreign or heterologous gene or genes. In this case, this gene shall be introduced into a different insertion site of the poxviral genome, and the second vector also differs in the poxvirus-homologous sequences directing the integration of the second foreign gene or genes into the genome of the poxvirus. After homologous recombination has occurred, the recombinant virus comprising two or more foreign or heterologous genes can be isolated. For introducing additional foreign genes into the recombinant virus, the steps of infection and transfection can be repeated by using the recombinant virus isolated in previous steps for infection and by using a further vector comprising a further foreign gene or genes for transfection.

Alternatively, the steps of infection and transfection as described above are interchangeable, i.e., a suitable cell can first be transfected by the plasmid vector comprising the foreign gene and, then, infected with the poxvirus. As a further alternative, it is also possible to introduce each foreign gene into different viruses, co-infect a cell with all the obtained recombinant viruses and screen for a recombinant including all foreign genes. A third alternative is ligation of DNA genome and foreign sequences in vitro and reconstitution of the recombined vaccinia virus DNA genome using a helper virus. A fourth alternative is homologous recombination in E. coli or another bacterial species between a vaccinia virus genome cloned as a bacterial artificial chromosome (BAC) and a linear foreign sequence flanked with DNA sequences homologous to sequences flanking the desired site of integration in the vaccinia virus genome.

The heterologous nucleic acid encoding one or more mosaic HIV antigens can be under the control of (i.e., operably linked to) one or more poxvirus promoters. In certain embodiments, the poxvirus promoter is a Pr7.5 promoter, a hybrid early/late promoter, a PrS promoter, a PrS5E promoter, a synthetic or natural early or late promoter, or a cowpox virus ATI promoter.

In certain embodiments of the invention, a first MVA vector expresses HIV antigens having SEQ ID NO: 1 and SEQ ID NO: 3, and a second MVA vector expresses HIV antigens having SEQ ID NO: 4 and either one of SEQ ID NO: 2 or SEQ ID NO: 12.

In other embodiments of the invention, a single MVA expresses HIV antigens having SEQ ID NO: 1, SEQ ID NO: 3, SEQ ID NO: 4, and either one of SEQ ID NO: 2 or SEQ ID NO: 12.

Immunogenic Compositions

Immunogenic compositions are compositions comprising an immunogenically effective amount of a purified or partially purified adenovirus 26 or MVA vector for use in the invention. The adenovirus 26 and MVA vectors can encode any mosaic HIV antigens in view of the present disclosure, and preferably encode one or more HIV antigens selected from the group consisting of SEQ ID NOs: 1-4 and 12. The one or more mosaic HIV antigens encoded by the adenovirus 26 vector can be the same or different as the one or more mosaic HIV antigens encoded by the MVA vector. Immunogenic compositions can be formulated as a vaccine, such as a primer vaccine or a booster vaccine, according to methods well known in the art. Such compositions can include adjuvants to enhance immune responses. The optimal ratios of each component in the formulation can be determined by techniques well known to those skilled in the art in view of the present disclosure.

As used herein, “an immunogenically effective amount” or “immunologically effective amount” means an amount of a composition or vector sufficient to induce a desired immune effect or immune response in a subject in need thereof. In one embodiment, an immunogenically effective amount means an amount sufficient to induce an immune response in a subject in need thereof, preferably a safe and effective immune response in a human subject in need thereof. In another embodiment, an immunogenically effective amount means an amount sufficient to produce immunity in a subject in need thereof, e.g., provide a therapeutic effect against a disease such as HIV infection. An immunogenically effective amount can vary depending upon a variety of factors, such as the physical condition of the subject, age, weight, health, etc. An immunogenically effective amount can readily be determined by one of ordinary skill in the art in view of the present disclosure.

An immunogenically effective amount can be administered in a single step (such as a single injection), or multiple steps (such as multiple injections), or in a single composition or multiple compositions. It is also possible to administer an immunogenically effective amount to a subject, and subsequently administer another dose of an immunogenically effective amount to the same subject, in a so-called prime-boost regimen. This general concept of a prime-boost regimen is well known to the skilled person in the vaccine field. Further booster administrations can optionally be added to the regimen, as needed.

As general guidance, an immunogenically effective amount when used with reference to a recombinant viral vector can range from about 10⁶ viral particles (vps) or plaque forming units (pfus) to about 10¹² viral particles or plaque forming units, for example 10⁶, 10⁷, 10⁸, 10⁹, 10¹⁰, 10¹¹, or 10¹² viral particles or plaque forming units.

In one embodiment, an immunogenic composition is a primer vaccine used for priming an immune response. According to embodiments of the invention, a primer vaccine comprises an immunogenically effective amount of one or more adenovirus 26 (Ad26) vectors together encoding four HIV antigens having the amino acid sequences of SEQ ID NO: 1, SEQ NO: 12, SEQ ID NO: 3 and SEQ ID NO: 4, and a pharmaceutically acceptable carrier. The HIV antigens can be encoded by the same Ad26 vector or different Ad26 vector, such as one, two, three, four or more Ad26 vectors.

The immunogenically effective amount of the one or more Ad26 vectors can be about 10⁸, 10⁹, 10¹⁰, 10¹¹, or 10¹² viral particles (vps), preferably about 10⁹ to 10¹¹ viral particles, and more preferably about 10¹⁰ viral particles, such as for instance about 0.5×10¹⁰, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, or 10×10¹⁰ viral particles. In certain embodiments, the immunogenically effective amount is about 5×10⁹ to about 1×10¹¹ viral particles, preferably about 5×10¹⁰ viral particles, such that the one or more Ad26 vectors are administered at a total dose of about 5×10⁹ to about 1×10¹¹ viral particles.

The immunogenically effective amount can be from one Ad26 vector or multiple Ad26 vectors. For example, a total administered dose of about 5×10⁹ to about 1×10¹¹ viral particles, such as for instance about 5×10¹⁰ viral particles, in the primer vaccine can be from four Ad26 vectors each encoding a different mosaic HIV antigen, such as those shown in SEQ ID NOs: 1, 12, 3, and 4.

In a particular embodiment, the immunogenically effective amount of Ad26 vectors together encoding SEQ ID NOs: 1, 12, 3, and 4 consists of four adenovirus vectors, namely a first Ad26 vector encoding the HIV antigen of SEQ ID NO: 1, a second Ad26 vector encoding the HIV antigen of SEQ ID NO: 12, a third Ad26 vector encoding the HIV antigen of SEQ ID NO: 3, and a fourth Ad26 vector encoding the HIV antigen of SEQ ID NO: 4.

In such embodiments where a primer vaccine comprises more than one Ad26 vector, the Ad26 vectors can be included in the composition in any ratio to achieve the desired immunogenically effective amount. Preferably, when the immunogenically effective amount of the Ad26 vectors consists of four Ad26 vectors, the first, second, third, and fourth Ad26 vectors are administered at a 1:1:1:1 ratio of viral particles (vps).

In another embodiment, an immunogenic composition is a booster vaccine. In some embodiments of the invention, a booster vaccine comprises an immunogenically effective amount of one or more adenovirus 26 (Ad26) vectors together encoding four HIV antigens having the amino acid sequences of SEQ ID NO: 1, SEQ NO: 12, SEQ ID NO: 3 and SEQ ID NO: 4, and a pharmaceutically acceptable carrier.

In those embodiments in which the booster vaccine comprises one or more Ad26 vectors, the immunogenically effective amount of the one or more Ad26 vectors in the booster vaccine is about 5×10⁹ to about 1×10¹¹ viral particles, preferably about 5×10¹⁰ viral particles, as described above with respect the primer vaccine. Preferably, the immunogenically effective amount of the one or more Ad26 vectors consists of four adenovirus vectors, namely a first Ad26 vector encoding the HIV antigen of SEQ ID NO: 1, a second Ad26 vector encoding the HIV antigen of SEQ ID NO: 12, a third Ad26 vector encoding the HIV antigen of SEQ ID NO: 3, and a fourth Ad26 vector encoding the HIV antigen of SEQ ID NO: 4, preferably administered at a 1:1:1:1 ratio of viral particles, also as described above with respect to the primer vaccine.

In other embodiments, a booster vaccine comprises an immunogenically effective amount of one or more MVA vectors together encoding four HIV antigens having the amino acid sequences of SEQ ID NO: 1, SEQ ID NO: 3, SEQ ID NO: 4, and either one of SEQ ID NO: 2 or SEQ ID NO: 12, and a pharmaceutically acceptable carrier. The HIV antigens expressed by MVA vectors can be encoded by the same MVA vector, or different MVA vectors, such as one, two, three, four or more MVA vectors.

The immunogenically effective amount of the one or more MVA vectors in the booster vaccine can be about 10⁶, 10⁷, 10⁸, 10⁹, or 10¹⁰ plaque forming units (pfus), preferably about 10⁷ to 10⁹ pfus, and more preferably about 10⁸ pfus, such as for instance about 0.5×10⁸, 1×10⁸, 2×10⁸, 3×10⁸, 4×10⁸, or 5×10⁸ pfus. In certain embodiments, the immunogenically effective amount is about 1×10⁷ to about 5×10⁸ pfus, preferably about 1×10⁸ pfus, such that the one or more MVA vectors are administered at a total dose of about 1×10⁷ to about 5×10⁸ pfus, preferably about 1'10⁸ pfus.

The immunogenically effective amount can be from one MVA vector or multiple MVA vectors. For example, in some embodiments, a total administered dose of about 1×10⁷ to about 5×10⁸ pfus, such as for instance about 1×10⁸ pfus, in the booster vaccine can be from two MVA vectors together encoding four HIV antigens having the amino acid sequences of SEQ ID NO: 1, SEQ ID NO: 3, SEQ ID NO: 4, and either one of SEQ ID NO: 2 or SEQ ID NO: 12. In other embodiments, a total administered dose of about 1×10⁷ to about 5×10⁸ pfus, such as for instance about 1×10⁸ pfus, in the booster vaccine can be from a single MVA vector encoding four HIV antigens having the amino acid sequences of SEQ ID NOs: 1, 3, and 4, and either one of SEQ ID NOs: 2 or 12.

In one particular embodiment, the immunogenically effective amount of MVA vectors together encoding SEQ ID NOs: 1, 3, and 4, and either one of SEQ ID NOs: 2 or 12 consists of two MVA vectors, namely a first MVA vector encoding the HIV antigens of SEQ ID NOs: 1 and 3, and a second MVA vector encoding the HIV antigens of SEQ ID NO: 4 and either one of SEQ ID NOs: 2 or 12. Preferably, when the immunogenically effective amount of the MVA vectors consists of two MVA vectors, the first and second MVA vectors are administered at a 1:1 ratio of pfus.

In another particular embodiment, the immunogenically effective amount of MVA vectors together encoding SEQ ID NOs: 1, 3, and 4, and either one of SEQ ID NOs: 2 or 12 consists of a single MVA vector encoding the HIV antigens of SEQ ID NOs: 1, 3, 4, and either one of SEQ ID NOs: 2 or 12.

In some embodiments of the invention, a booster vaccine is administered in combination with one or more isolated HIV gp140 envelope polypeptides. According to embodiments of the invention, when used with reference to the total amount of the one or more isolated HIV envelope polypeptides administered as part of a boosting immunization, such as at least one of the isolated HIV gp140 envelope polypeptides having the amino acid sequences of SEQ ID NO: 9 (clade C gp140 polypeptide) and SEQ ID NO: 10 (mosaic gp140 polypeptide), an immunogenically effective amount can range from, e.g. about 125 μg to 350 e.g. about 125, 150, 200, 250, 300, or 350 μg of the one or more isolated HIV envelope polypeptides. In certain embodiments, a first booster vaccine composition comprising one or more Ad26 vectors or one or more MVA vectors is administered in combination with a second booster vaccine composition comprising two isolated HIV envelope gp140 polypeptides, one clade C gp140 polypeptide having the amino acid sequence of SEQ ID NO: 9 and one mosaic gp140 polypeptide having the amino acid sequence of SEQ ID NO: 10, each one for instance present in about 125 μg per administration to a total of about 250 μg.

The preparation and use of immunogenic compositions are well known to those of ordinary skill in the art. Liquid pharmaceutical compositions generally include a liquid carrier such as water, petroleum, animal or vegetable oils, mineral oil or synthetic oil. Physiological saline solution, dextrose or other saccharide solution or glycols, such as ethylene glycol, propylene glycol or polyethylene glycol can also be included. The immunogenic compositions used in the invention, e.g., primer vaccines and booster vaccines, can be formulated for administration according to any method known in the art in view of the present disclosure, and are preferably formulated for intramuscular administration.

The priming and/or boosting compositions of the invention can comprise other antigens. The other antigens used in combination with the adenovirus 26 and/or MVA vectors are not critical to the invention and can be, for example, other HIV antigens and nucleic acids expressing them.

The immunogenic compositions useful in the invention can further optionally comprise adjuvants. Adjuvants suitable for co-administration in accordance with the invention should be ones that are potentially safe, well tolerated and effective in people. Non-limiting examples include QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, Aluminum salts such as Aluminum Phosphate (e.g. AdjuPhos) or Aluminum Hydroxide, and MF59.

For example, a preferred adjuvant for administration together with isolated HIV envelope polypeptides is aluminum phosphate. According to embodiments of the invention, when used with reference to the total amount of aluminum phosphate in a boosting composition comprising one or more HIV envelope polypeptides, the total amount of aluminum phosphate administered can range from, e.g. about 10 μg to about 1000 μg, e.g. about 200 μg to 650 μg, e.g. about 200, 250, 300, 350, 400, 425, 450, 475, 500, 550, or 600 μg, preferably about 425 μg of aluminum phosphate.

The immunogenic compositions used for priming and boosting an immune response according to embodiments of the invention comprise a pharmaceutically acceptable carrier, such as a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g., intramuscular, subcutaneous, oral, intradermal, cutaneous, intramucosal (e.g., gut), intranasal or intraperitoneal routes. Preferably, the pharmaceutically acceptable carrier included in the priming and boosting compositions of the invention is suitable for intramuscular administration.

Method of Inducing an Immune Response Against HIV Infection

The priming and boosting vaccine compositions according to embodiments of the invention can be used in the methods of the invention described herein. The methods of the invention relate to inducing an immune response against a human immunodeficiency virus (HIV) in an HIV-infected subject undergoing antiretroviral therapy. The methods of priming and boosting an immune response according to embodiments of the invention are effective to induce an immune response against one or multiple clades of HIV.

In one general aspect, a method of inducing an immune response against a human immunodeficiency virus (HIV) in an HIV-infected human subject undergoing antiretroviral therapy (ART) comprises administering to the human subject:
(i) a primer vaccine comprising an immunogenically effective amount of one or more adenovirus 26 (Ad26) vectors together encoding four HIV antigens having the amino acid sequences of SEQ ID NO: 1, SEQ ID NO: 12, SEQ ID NO: 3, and SEQ ID NO: 4, and a pharmaceutically acceptable carrier; and
(ii) a booster vaccine comprising an immunogenically effective amount of one or more Modified Vaccinia Ankara (MVA) vectors together encoding four HIV antigens having the amino acid sequences of SEQ ID NO: 1, SEQ ID NO: 3, SEQ ID NO: 4 and either one of SEQ ID NO: 2 or SEQ ID NO: 12, and a pharmaceutically acceptable carrier.

In some embodiments of this aspect of the invention, the method further comprises administering to the human subject one or more isolated HIV gp140 envelope polypeptides in combination with the booster vaccine. In such embodiments, the method preferably further comprises administering to the human subject at least one isolated HIV gp140 envelope polypeptide selected from the group consisting of two trimeric HIV gp140 envelope polypeptides having the amino acid sequences of SEQ ID NO: 9 and SEQ ID NO :10, in combination with the booster vaccine.

In another general aspect, a method of inducing an immune response against a human immunodeficiency virus (HIV) in an HIV-infected human subject undergoing antiretroviral therapy (ART) comprises administering to the human subject:
(i) a primer vaccine comprising an immunogenically effective amount of one or more adenovirus 26 (Ad26) vectors together encoding four HIV antigens having the amino acid sequences of SEQ ID NO: 1, SEQ ID NO: 12, SEQ ID NO: 3, and SEQ ID NO: 4, and a pharmaceutically acceptable carrier; and
(ii) a booster vaccine comprising:
   (ii,a) a first booster vaccine composition comprising an immunogenically effective amount of one or more adenovirus 26 (Ad26) vectors together encoding four HIV antigens having the amino acid sequences of SEQ ID NO: 1, SEQ ID NO: 12, SEQ ID NO: 3, and SEQ ID NO: 4, and a pharmaceutically acceptable carrier; and
   (ii,b) a second booster vaccine composition comprising at least one, preferably two, isolated HIV gp140 envelope polypeptide having an amino acid sequence selected from the group consisting of SEQ ID NO: 9 and SEQ ID NO: 10, preferably together with an aluminum phosphate adjuvant, and a pharmaceutically acceptable carrier,
      wherein the first and second booster vaccine compositions are administered in combination.

Any of the primer and booster vaccine compositions described herein can be used in a method of inducing an immune response against HIV according to the invention. Embodiments of the primer vaccine; booster vaccine; Ad26 vectors; MVA vectors; HIV antigens encoded by the Ad26 and MVA vectors; isolated gp140 polypeptide, etc. that can be used in the methods of the invention are discussed in detail above and in the illustrative examples below.

According to embodiments of the invention, “inducing an immune response” when used with reference to the methods described herein encompasses causing a desired immune response or effect in a subject in need thereof against an HIV infection, preferably for therapeutic purposes. “Inducing an immune response” also encompasses providing a therapeutic immunity for treating against a pathogenic agent, i.e., HIV. As used herein, the term “therapeutic immunity” or “therapeutic immune response” means that the HIV-infected vaccinated subject is able to control an infection with the pathogenic agent, i.e., HIV, against which the vaccination was done. In one embodiment, “inducing an immune response” means producing an immunity in a subject in need thereof, e.g., to provide a therapeutic effect against a disease such as HIV infection. In certain embodiments, “inducing an immune response” refers to causing or improving cellular immunity, e.g., T cell response, against HIV. In certain embodiments, “inducing an immune response” refers to causing or improving a humoral immune response against HIV. In certain embodiments, “inducing an immune response” refers to causing or improving a cellular and a humoral immune response against HIV. Typically, the administration of the primer and booster vaccine compositions according to embodiments of the invention will have a therapeutic aim to generate an immune response against HIV after HIV infection or development of symptoms characteristic of HIV infection.

The patient population for treatment according to the methods of the invention described herein is HIV-infected human subjects, particularly HIV-infected human subjects undergoing antiretroviral therapy (ART). The terms “HIV infection” and “HIV-infected” as used herein refer to invasion of a human host by HIV. As used herein, “an HIV-infected human subject” refers to a human subject in whom HIV has invaded and subsequently replicated and propagated within the human host, thus causing the human host to be infected with HIV or have an HIV infection or symptoms thereof. An “HIV-infected human subject” has been diagnosed with HIV infection, i.e., tests positive in a screen for HIV infection, e.g. using any assay that is US FDA-approved.

As used herein, “undergoing antiretroviral therapy” refers to a human subject, particularly an HIV-infected human subject, that is being administered, or who has initiated treatment with antiretroviral drugs. According to embodiments of the invention, the antiretroviral therapy (ART) is started prior to the first administration of the primer vaccine, for instance, about 2 to 6 weeks prior, such as about 2, 3, 4, 5, or 6 weeks prior, or 2-48 months prior, such as about 2, 3, 5, 6, 8, 12, 16, 20, 24, 30, 36, 42, or 48 months prior, or longer. In certain embodiments the ART is started earlier than about 44-52 weeks, preferably earlier than about 48 weeks prior to the first administration of the primer vaccine. In a subject undergoing antiretroviral therapy, the antiretroviral therapy is continued during administration of the prime/boost vaccine regimen of the invention. ART is considered “suppressive” as used herein if the subject has plasma HIV RNA levels at less than 50 copies/mL for a certain period of time, including the possibility of blips. The term “stable suppressive” ART as used herein means that the suppressive ART regimen is not modified for a certain period of time.

In certain embodiments, a human subject undergoing antiretroviral therapy is on current stable suppressive ART for at least twenty-four weeks, meaning that while receiving the same ART regimen the subject has plasma HIV ribonucleic acid (RNA) levels at less than 50 copies/mL for at least 24 weeks prior to initiation of a prime/boost vaccine regimen according to the invention. However, the human subject can have one or more blips (i.e., instances) of plasma HIV RNA greater than 50 copies/ml to less than 200 copies/ml within this period, such as within the 24 week period prior to the initiation of a prime/boost vaccine regimen, provided that screening immediately prior to initiation of the prime/boost vaccine regimen is less than 50 copies/ml.

An HIV-infected subject can initiate ART during the acute phase of HIV infection, or outside of the acute phase of HIV infection. In a preferred embodiment, the subject initiated ART outside the acute phase of HIV infection. The term “acute HIV infection” refers to the initial stage of HIV infection. In general, there are three stages of HIV infection: (1) acute HIV infection, (2) clinical latency, and (3) acquired immunodeficiency syndrome (AIDS). During acute HIV infection, the host typically develops symptoms such as fever, swollen glands, sore throat, rash, muscle and joint aches and pains, headache, etc., as a result of the body's natural response to the HIV infection. During the acute stage of infection, large amounts of the HIV virus are produced in the host, and CD4 levels can decrease rapidly, because the HIV uses CD4 to replicate and then subsequently destroys the CD4. Once the natural immune response of the host brings the level of HIV in the host to a stable level, also known as viral set point, CD4 count begins to increase, but likely not to pre-infection levels. Acute HIV infection is also characterized as Fiebig stages I, II, III, and IV as described in Fiebig et al., “Dynamics of HIV viremia and antibody seroconversion in plasma donors: implications for diagnosis and staging of primary HIV infection.” AIDS (London, England) (2003) 17(13) 1871-1879, which is herein incorporated by reference in its entirety.

Acute HIV infection is typically within two to four weeks after a host is exposed to and infected with HIV and continues for an additional two to four weeks. The acute HIV infection stage lasts until the host creates its own antibodies against HIV, at which point the clinical latency stage begins. During the clinical latency stage, HIV is living or developing in the host without causing any symptoms, or only causing mild symptoms. HIV reproduces at very low levels during the clinical latency stage, although the HIV is still active. The clinical latency stage is sometimes also referred to as “chronic HIV infection” or “asymptomatic HIV infection.” Chronic HIV infection is characterized as Fiebig stage VI.

Chronic HIV infection (i.e., Fiebig stage VI) typically begins about 100 days (i.e., about 14 weeks) after a host is exposed to and infected with HIV. A subject infected with HIV that has progressed to Fiebig stage VI can be referred to or described as a “chronically-infected subject,” “a chronic HIV-infected subject,” or “a subject having chronic HIV infection.” A subject initiating ART outside of the acute or early phase of HIV infection is one who has not begun ART before entering Fiebig VI stage. Whether or not a subject has initiated ART prior to entering Fiebig VI stage of HIV infection can be determined by a clinician based on the subject's available medical history and laboratory data at the time of HIV diagnosis.

According to embodiments of the invention, a subject who initiates ART outside of the acute phase of HIV infection (i.e., during chronic HIV infection) begins treatment with antiretroviral drugs at the earliest at about 12-16 weeks, after being exposed to and infected with HIV, such as about 12, 13, 14, 15, or 16 weeks or later, after exposure and infection with HIV. In contrast, a subject who initiates ART during acute HIV infection typically begins treatment with antiretroviral drugs at or prior to about 2 weeks to about 8 weeks after being exposed to and infected with HIV, such as about 1, 2, 3, 4, 5, 6, 7, or 8 weeks after exposure and infection. Thus, chronic HIV infection is thought to be more difficult to treat than acute HIV infection, at least because a chronically infected HIV subject typically has larger HIV viral reservoirs than an acutely infected subject due to the longer period of infection prior to initiating any treatment. Subjects who began ART during acute HIV infection and have plasma HIV RNA levels of less than 50 copies/ml for at least 24 weeks, preferably at least 48 weeks, have low HIV viral reservoirs and/or lower involvement of their viral reservoirs with HIV, and therefore have a higher chance for maintained viral suppression in the absence of ART, i.e., HIV remission. However, due to the differences in progression of acute and chronic HIV infection, it is not certain as to whether therapies effective to treat acute HIV infection will likewise be effective to treat chronic HIV infection.

In some embodiments of the invention, the HIV-infected subject is a chronically HIV-infected subject. A chronically HIV-infected subject can initiate ART at any phase of infection, such as during the acute phase of HIV infection or outside the acute phase of HIV infection. Preferably, a chronically HIV-infected subject initiates ART outside of the acute phase of HIV infection.

A subject undergoing ART can be administered or treated with any antiretroviral drugs known in the art in view of the present disclosure. ART are medications that treat HIV, although the drugs do not kill the virus or remove the virus from the body. However, when taken in combination they can prevent the growth of the virus. When the virus is slowed down, so is HIV disease. Antiretroviral drugs are referred to as ARV. Combination ARV therapy (cART) is referred to as highly active ART (HAART). Typically, an ART regimen includes at least three antiviral compounds, e.g., two different reverse transcriptase inhibitors plus either a non-nucleoside reverse transcriptase inhibitor or protease inhibitor or integrase inhibitor.

One of ordinary skill in the art will be able to determine the appropriate antiretroviral treatment, frequency of administration, dosage of the ART, etc. so as to be compatible with simultaneous administration of the prime/boost vaccine regimens of the invention. Examples of antiretroviral drugs used for ART include, but are not limited to nucleoside reverse transcriptase inhibitors (NRTIs, non-limiting examples of which include zidovudine, didanosine, stavudine, lamivudine, abacavir, tenofovir, combivir [combination of zidovudine and lamivudine], trizivir [combination of zidovudine, lamivudine and abacavir], emtricitabine, truvada [combination of emtricitabine and tenofovir], and epzicom [combination of abacavir and lamivudine]), non-nucleoside reverse transcriptase inhibitors (NNRTIs, non-limiting examples of which include nevirapine, delavirdine, efavirenz, etravirine, and rilpivirine), protease inhibitors (PIs, non-limiting examples of which include saquinavir, indinavir, ritonavir, nelfinavir, amprenavir, lopinavir/ritonavir, atazanavir, fosamprenavir, tipranavir, darunavir), integrase inhibitors (INSTIs, non-limiting examples including raltegravir, elvitegravir, and dolutegravir), and fusion inhibitors, entry inhibitors and/or chemokine receptor antagonists (FIs, CCRS antagonists; non-limiting examples including enfuvirtide, aplaviroc, maraviroc, vicriviroc, and cenicriviroc).

According to embodiments of the invention, the booster vaccine is first administered after the primer vaccine is first administered. In certain embodiments of the invention, the booster vaccine is first administered at about 12-52 weeks, e.g. about 16-32, e.g. about 22-26, e.g. about 24 weeks, after the primer vaccine is initially administered. One of ordinary skill in the art will be able to vary the exact timing of the priming and boosting vaccines, frequency of administration thereof, dosage thereof, etc., based upon the teachings herein and clinical experience.

According to embodiments of the invention, a primer vaccine is administered at least once and a booster vaccine at least once. According to embodiments of the invention, the booster vaccine is first administered at about 22-26 weeks, such as 22, 23, 24, 25, or 26 weeks after the primer vaccine is initially administered. In certain embodiments, the booster vaccine is first administered at about 24 weeks after the primer vaccine is initially administered.

In some preferred embodiments, the primer vaccine is re-administered after the primer vaccine is initially administered, and in such embodiments preferably re-administered before the booster vaccine is first administered. For example, the primer vaccine can be re-administered at about 10-14 weeks after the primer vaccine is initially administered, such as about 10, 11, 12, 13, or 14 weeks after the primer vaccine is initially administered, preferably at about 12 weeks after the primer vaccine is initially administered.

In other preferred embodiments, the booster vaccine is re-administered after the booster vaccine is first administered. In certain embodiments, the booster vaccine is first administered at about 22 to 26 weeks, such as 22, 23, 24, 25, or 26 weeks after the primer vaccine is initially administered, preferably at about 24 weeks after the primer vaccine is initially administered. The booster vaccine can in certain embodiments be re-administered at about 34 to 38 weeks, such as 34, 35, 36, 37, or 38 weeks, or alternatively at about 44 to 52 weeks, such as 46, 47, 48, 49, or 50 weeks, after the primer vaccine is initially administered. In certain preferred embodiments, the booster vaccine is re-administered at about 36 weeks after the primer vaccine is initially administered.

In particular embodiments of the invention, both the primer vaccine and the booster vaccine are re-administered to the subject. The primer vaccine can be re-administered at about 10-14 weeks, such as for instance about 12 weeks after the primer vaccine is initially administered; and the booster vaccine can be re-administered at about 34 to 38 weeks, such as for instance about 36 weeks after the primer vaccine is initially administered.

Further booster administrations are possible, and embodiments of the disclosed methods also contemplate administration of such additional boosting immunizations with immunogenic compositions containing Ad26 vectors, MVA vectors, and/or HIV gp140 polypeptides. Any of the Ad26 vectors, MVA vectors, and HIV gp140 polypeptides described herein can be used in additional boosting immunizations.

The primer and booster vaccine compositions can be administered by any method known in the art in view of the present disclosure, and administration is typically via intramuscular, intradermal or subcutaneous administration, preferably intramuscular administration. Intramuscular administration can be achieved by using a needle to inject a suspension or solution of the adenovirus and/or MVA vectors, and/or gp140 polypeptides. An alternative is the use of a needleless injection device to administer the composition (using, e.g., Biojector™) or a freeze-dried powder containing the vaccine.

Other modes of administration, such as intravenous, cutaneous, intradermal, oral, intratracheal, or nasal are also envisaged as well. For intravenous, cutaneous or subcutaneous injection, the vector will be in the form of a parenterally acceptable aqueous solution which is pyrogen-free and has suitable pH, isotonicity and stability. Those of ordinary skill in the art are well able to prepare suitable solutions using, for example, isotonic vehicles such as Sodium Chloride Injection, Ringer's Injection, and Lactated Ringer's Injection. Preservatives, stabilizers, buffers, antioxidants and/or other additives can be included, as required. A slow-release formulation can also be employed.

In certain embodiments, a method of inducing an immune response according to the invention further comprises administering a latent viral reservoir purging agent. Cells latently infected with HIV carry integrated virus that is transcriptionally silent, making it difficult to effectively eradicate HIV infection in treated subjects. As used herein, “reservoir purging agent” and “latent viral reservoir purging agent” refer to a substance that reduces the latent pool of HIV by reactivating HIV reservoirs, such as by inducing expression of quiescent HIV. Examples of latent viral reservoir purging agents suitable for use with the invention include, but are not limited to, histone deacetylase (HDAC) inhibitors and modulators of toll-like receptors (e.g., TLR7), such as those described in WO2016/007765 and WO2016/177833, which are herein incorporated by reference in their entireties. The latent viral reservoir purging agent can be administered before, after, or co-administered (i.e., administered in combination) with one or more of the priming and boosting immunizations described herein. The vaccination of a combination of adenovirus 26 vectors encoding Gag, Pol and Env antigens as a prime, followed by MVA vectors encoding such antigens as a boost, in combination with TLR7 stimulation has shown to result in improved virologic control and delayed viral rebound following discontinuation of antiretroviral therapy in rhesus monkey model studies, demonstrating the potential of therapeutic vaccination combined with innate immune stimulation to aim at a functional cure for HIV infection (Borducchi E. N., et al, 2016, Nature 540: 284-287 (doi: 10/1038/nature20583)), the content of which is incorporated herein by reference in its entirety.

In other embodiments, subjects undergo interruption (also referred to as discontinuation, used interchangeably herein) of ART after completion of the vaccine regimen according to embodiments of the invention. In some embodiments, subjects can undergo antiretroviral analytical treatment interruption (ARV ATI) after completion of vaccine regimen according to embodiments of the invention. “Antiretroviral analytical treatment interruption” and “ARV ATI” as used in the invention refer to discontinuation of treatment with antiretroviral drugs in order to assess viral suppression and viremic control in the absence of continued ART. Typically, subjects can undergo ARV ATI, i.e., ART can be discontinued, for example when the subject has plasma HIV RNA levels at less than 50 copies/mL for at least about 52 weeks, but a subject can still undergo ARV ATI even if the subject has one or more blips (i.e., instances) of plasma HIV RNA greater than 50 copies/ml to less than 200 copies/ml within this period, provided that the screening immediately prior to ARV ATI shows less than 50 copies/ml of plasma HIV RNA.

According to embodiments of the invention, the ART can be stopped at about 10-14 weeks, such as 10, 11, 12, 13, or 14 weeks after the last booster vaccine is administered. In certain embodiments, the last booster vaccine is administered at about 34-38 weeks after the primer vaccine is initially administered. In these embodiments, the ART can be stopped at about 46 to 50 weeks, such as 46, 47, 48, 59, or 60 weeks after the primer vaccine is initially administered, and preferably about 60 weeks after the primer vaccine is initially administered. In other embodiments, for subjects who are on non-nucleoside reverse transcriptase inhibitor (NNRTI)-based ART, a boosted protease inhibitor can be administered in place of the NNRTI for about 1-2 weeks prior to stopping ART to reduce the risk of developing NNRTI resistance. It is also possible to administer an activator (e.g. a histone deacetylase inhibitor or TLR7 modulator) during the ATI stage to activate any (e.g. latent) HIV reservoir and thereby improve the immune response.

Subjects undergoing ARV ATI can be monitored, e.g., by measuring plasma HIV RNA levels. As a non-limiting example, monitoring after the initiation of ARV ATI can occur up to two times per week during the first six weeks when rebound viremia is most likely to occur. “Rebound viremia” is for example defined as plasma HIV RNA levels of greater than 1,000 copies/ml after ARV ATI. ART can be re-initiated in subjects with rebound viremia. Preferably, a subject treated according to the methods of the invention will maintain viremic control after ART interruption. As used herein, “maintain viremic control” is in exemplary embodiments defined as at least 24 weeks with plasma HIV RNA of less than 50 copies/mL after ARV ATI. The “maintained viremic control” criterion is in certain exemplary embodiments still deemed to be met if there are one or more instances of plasma HIV RNA greater than 50 copies/ml to less than 1000 copies/ml, as long as the subject does not have plasma HIV RNA levels above 1000 copies/ml on two consecutive determinations at least one week apart.

Typically (not using the methods of the instant invention) human HIV-infected subjects have a return of viremia after 2-3 weeks following ART interruption. Without wishing to be bound by any theories, it is believed that vaccine therapy using the prime/boost vaccine compositions according to embodiments of the invention among individuals with fully suppressed HIV will result in a measurable immune response and maintain viremic control after ARV ATI. In some embodiments, subjects can discontinue ART after being treated according to a method of the invention. Discontinuation of ART can be for long periods of time (e.g., at least 24 weeks, preferably longer, e.g. at least about 28, 32, 36, 40, 44, 48, 52 weeks, 16 months, 18, 20, 22, 24 months, or even longer). Such periods of time in which ART is stopped or discontinued are referred to as a “holiday” or “ART holiday” or “treatment holiday”. In other embodiments, vaccine therapy according to the methods of the invention can provide HIV remission, meaning that viral suppression is maintained in the absence of ART. In certain embodiments of the invention, a human subject that received the priming and boosting vaccines of the invention, discontinues ART and maintains viral suppression for at least 24 weeks after discontinuing ART.

In one exemplary regimen of the invention, a primer vaccine comprising one or more adenovirus 26 vectors is administered (e.g., intramuscularly) in an amount of about 100 μl to about 2 ml, preferably about 0.5 ml, of a solution containing concentrations of about 10⁸ to 10¹² virus particles/ml. The initial primer vaccination is followed by a booster vaccine comprising one more MVA vectors administered (e.g., intramuscularly) in an amount of about 100 μl to about 2 ml, preferably about 0.5 ml, of a solution containing concentrations of about 10⁶ to 10⁹ pfu/ml.

In another exemplary regimen of the invention, a primer vaccine comprising one or more adenovirus 26 vectors is administered (e.g., intramuscularly) in an amount of about 100 μl to about 2 ml, preferably about 0.5 ml, of a solution containing concentrations of about 10⁸ to 10¹² virus particles/ml. The initial primer vaccination is followed by a booster vaccine comprising one more adenovirus 26 vectors administered (e.g., intramuscularly) in an amount of about 100 μl to about 2 ml, preferably about 0.5 ml, of a solution containing concentrations of about 10⁸ to 10¹² virus particles/ml in combination with one or more isolated HIV gp140 polypeptides in an amount of about 100 μl to about 2 ml, preferably about 0.5 ml, of a solution, to a total dose per administration of about 250 mg polypeptide and aluminum phosphate adjuvant (425 microgram (μg) aluminum per dose).

The skilled artisan (e.g., practitioner) will appreciate that certain factors may influence the dosage required to effectively treat a subject, including but not limited to the severity of the disease or disorder, previous treatments, the general health and/or age of the subject, and other diseases present.

The invention also relates to a vaccine combination for use in inducing an immune response against a human immunodeficiency virus (HIV) in an HIV-infected human subject undergoing antiretroviral therapy (ART), wherein the vaccine combination comprises a primer vaccine and a booster vaccine according to embodiments of the invention. The invention yet further relates to use of a vaccine combination in the manufacture of a medicament for inducing an immune response against a human immunodeficiency virus (HIV) in an HIV-infected human subject undergoing antiretroviral therapy (ART), wherein the vaccine combination comprises a primer vaccine and a booster vaccine according to embodiments of the invention. All aspects and embodiments of the invention as described herein with respect to methods of inducing an immune response against a human immunodeficiency virus (HIV) in an HIV-infected human subject undergoing antiretroviral therapy (ART) can be applied to the vaccine combinations for use and/or uses of the vaccine combination in the manufacture of a medicament for inducing an immune response against HIV in an HIV-infected subject undergoing ART.

### Embodiments

Embodiment 1 is a method of inducing an immune response against a human immunodeficiency virus (HIV) in an HIV-infected human subject undergoing antiretroviral therapy (ART), the method comprising administering to the human subject:
(i) a primer vaccine comprising an immunogenically effective amount of one or more adenovirus 26 (Ad26) vectors together encoding four HIV antigens having the amino acid sequences of SEQ ID NO: 1, SEQ ID NO: 12, SEQ ID NO: 3, and SEQ ID NO: 4, and a pharmaceutically acceptable carrier; and
(ii) a booster vaccine comprising an immunogenically effective amount of one or more Modified Vaccinia Ankara (MVA) vectors together encoding four HIV antigens having the amino acid sequences of SEQ ID NO: 1, SEQ ID NO: 3, and SEQ ID NO: 4, and either one of SEQ ID NO: 2 or SEQ ID NO: 12, and a pharmaceutically acceptable carrier.

Embodiment 2 is the method of embodiment 1, wherein the immunogenically effective amount of the one or more Ad26 vectors encoding the four HIV antigens consists of a first Ad26 vector encoding the HIV antigen of SEQ ID NO: 1, a second Ad26 vector encoding the HIV antigen of SEQ ID NO: 12, a third Ad26 vector encoding the HIV antigen of SEQ ID NO: 3, and a fourth Ad26 vector encoding the HIV antigen of SEQ ID NO: 4, preferably the first, second, third and fourth Ad26 vectors are administered at a 1:1:1:1 ratio of viral particles (vps).

Embodiment 3 is the method of embodiment 1 or embodiment 2, wherein the immunogenically effective amount of the one or more MVA vectors encoding the four HIV antigens consists of a single MVA vector encoding the HIV antigens of SEQ ID NOs: 1, 3, 4, and either one of SEQ ID NOs: 2 or 12.

Embodiment 4 is the method of embodiment 1 or embodiment 2, wherein the immunogenically effective amount of the one or more MVA vectors encoding the four HIV antigens consists of a first MVA vector encoding the HIV antigens of SEQ ID NOs: 1 and 3, and a second MVA vector encoding the HIV antigen of SEQ ID NO: 4 and either one of the HIV antigens of SEQ ID NOs: 2 or 12, preferably the first and second MVA vectors are administered at a 1:1 ratio of plaque-forming units (pfu).

Embodiment 5 is the method of embodiment 2, 3 or 4, wherein the first, second, third, and fourth Ad26 vectors together are administered at a total dose of about 5×10⁹ to about 1×10¹¹ viral particles (vp), preferably about 5×10¹⁰ vp, of the Ad26 vectors.

Embodiment 6 is the method of any one of embodiments 3 to 5, wherein the single MVA vector or the first and second MVA vectors together are administered at a total dose of about 1×10⁷ to about 5×10⁸ plaque-forming units (pfu), preferably about 1×10⁸ pfu, of the MVA vector or vectors.

Embodiment 7 is the method of any one of embodiments 1 to 6, further comprising administering to the human subject one or more isolated HIV gp140 envelope polypeptides and preferably together with an aluminum phosphate adjuvant, in combination with the booster vaccine.

Embodiment 8 is the method of embodiment 7, wherein the one or more isolated HIV gp140 envelope polypeptides is at least one of isolated HIV gp140 envelope polypeptides having the amino acid sequences of SEQ ID NO: 9 and SEQ ID NO: 10.

Embodiment 9 is the method of embodiment 8, wherein the one or more isolated HIV gp140 envelope polypeptides consists of two trimeric HIV gp140 envelope polypeptides having the amino acid sequences of SEQ ID NO: 9 and SEQ ID NO: 10, preferably at a 1:1 ratio by weight.

Embodiment 10 is a method of inducing an immune response against a human immunodeficiency virus (HIV) in an HIV-infected human subject undergoing antiretroviral therapy (ART), the method comprising administering to the human subject: a primer vaccine comprising an immunogenically effective amount of one or more adenovirus 26 (Ad26) vectors together encoding four HIV antigens having the amino acid sequences of SEQ ID NO: 1, SEQ ID NO: 12, SEQ ID NO: 3, and SEQ ID NO: 4, and a pharmaceutically acceptable carrier, in a total dose of about 5×10⁹ to about 1×10¹¹ viral particles (vp), preferably about 5×10¹⁰ vp, of the Ad26 vectors; and
(ii) a booster vaccine comprising:
   (ii,a) a first booster vaccine composition comprising an immunogenically effective amount of one or more adenovirus 26 (Ad26) vectors together encoding four HIV antigens having the amino acid sequences of SEQ ID NO: 1, SEQ ID NO: 12, SEQ ID NO: 3, and SEQ ID NO: 4, and a pharmaceutically acceptable carrier, and
   (ii,b) a second booster vaccine composition comprising at least one isolated HIV gp140 envelope polypeptide having an amino acid sequence selected from the group consisting of SEQ ID NO: 9 and SEQ ID NO: 10, an aluminum phosphate adjuvant and a pharmaceutically acceptable carrier,
      wherein the first and second booster vaccine compositions are administered in combination.

Embodiment 11 is the method of embodiment 10, wherein the immunogenically effective amount of the one or more Ad26 vectors encoding the four HIV antigens consists of a first Ad26 vector encoding the HIV antigen of SEQ ID NO: 1, a second Ad26 vector encoding the HIV antigen of SEQ ID NO: 12, a third Ad26 vector encoding the HIV antigen of SEQ ID NO: 3, and a fourth Ad26 vector encoding the HIV antigen of SEQ ID NO: 4, preferably administered at a 1:1:1:1 ratio of vps.

Embodiment 12 is the method of embodiment 11, wherein the first, second, third, and fourth Ad26 vectors together are administered at a total dose of about 5×10⁹ to about 1×10¹¹ viral particles (vp), preferably about 5×10¹⁰ vp, of the Ad26 vectors.

Embodiment 13 is the method of any one of embodiments 10 to 12, wherein the at least one of the isolated HIV gp140 envelope polypeptides consists of two trimeric HIV gp140 envelope polypeptides having the amino acid sequences of SEQ ID NO: 9 and SEQ ID NO: 10, preferably at a 1:1 ratio by weight.

Embodiment 14 is the method of any one of embodiments 10 to 13, wherein the total dose of the at least one isolated HIV gp140 envelope polypeptide is about 125 μg to 350 μg, preferably about 250 μg.

Embodiment 15 is the method of any one of embodiments 1 to 14, wherein the booster vaccine is administered at about 22-26 weeks after the primer vaccine is initially administered.

Embodiment 16 is the method of any one of embodiments 1 to 15, further comprising re-administering the primer vaccine at about 10-14 weeks after the primer vaccine is initially administered; and re-administering the booster vaccine at about 34 to 38 weeks after the primer vaccine is initially administered.

Embodiment 17 is the method of embodiment 16, wherein the primer vaccine is re-administered at about 12 weeks after the primer vaccine is initially administered; the booster vaccine is first administered at about 24 weeks after the primer vaccine is initially administered; and the booster vaccine is re-administered at about 36 weeks after the primer vaccine is initially administered.

Embodiment 18 is the method according to any one of embodiments 1 to 17, wherein the primer vaccine and booster vaccine are administered via intramuscular injection.

Embodiment 19 is the method of any one of embodiments 1 to 18, wherein the human subject has initiated ART outside of the acute phase of HIV infection.

Embodiment 20 is the method of any one of embodiments 1 to 19, wherein the human subject is a chronically HIV-infected subject.

Embodiment 21 is the method of any one of embodiments 1 to 20, wherein the subject is on suppressive ART for at least 48 weeks prior to the initial administration of the primer vaccine.

Embodiment 22 is the method of embodiment 21, wherein the subject is on current stable suppressive ART at least 24 weeks prior to the initial administration of the primer vaccine.

Embodiment 23 is the method of embodiment 22, wherein the subject has sustained viremic control defined as plasma HIV RNA of less than 50 copies per ml for at least 24 weeks prior to the initial administration of the primer vaccine, optionally with one or more blips of plasma HIV RNA greater than 50 copies/ml to less than 200 copies/ml, provided that screening immediately prior to the initial administration of the primer vaccine is less than 50 copies/ml.

Embodiment 24 is the method of any one of embodiments 1 to 23, further comprising administering to the subject a reservoir purging agent.

Embodiment 25 is the method of embodiment 24, wherein the reservoir purging agent is a toll-like receptor 7 (TLR7) agonist or a histone deacetylase (HDAC) inhibitor, preferably a TLR7 agonist.

Embodiment 26 is the method of any one of embodiments 1-25, wherein the ART is discontinued at about 10-14 weeks after the last booster vaccine is administered.

Embodiment 27 is the method of embodiment 26, wherein the subject has sustained viremic control after discontinuing ART.

Embodiment 28 is the method of embodiments 1-27, wherein administration of the primer vaccine and booster vaccine induces an immune response against multiple clades of HIV in the subject.

Embodiment 30 is a vaccine combination for use in inducing an immune response against a human immunodeficiency virus (HIV) in an HIV-infected subject undergoing antiretroviral therapy (ART), wherein the vaccine combination comprises:
(i) a primer vaccine comprising an immunogenically effective amount of one or more adenovirus 26 (Ad26) vectors together encoding four HIV antigens having the amino acid sequences of SEQ ID NO: 1, SEQ ID NO: 12, SEQ ID NO: 3, and SEQ ID NO: 4, and a pharmaceutically acceptable carrier; and
(ii) a booster vaccine comprising an immunogenically effective amount of one or more Modified Vaccinia Ankara (MVA) vectors together encoding four HIV antigens having the amino acid sequences of SEQ ID NO: 1, SEQ ID NO: 3, and SEQ ID NO: 4, and either one of SEQ ID NO: 2 or SEQ ID NO: 12, and a pharmaceutically acceptable carrier.

Embodiment 31 is use of a vaccine combination in the preparation or manufacture of a medicament for inducing an immune response against a human immunodeficiency virus (HIV) in an HIV-infected subject undergoing antiretroviral therapy (HIV), wherein the vaccine combination comprises:
(i) a primer vaccine comprising an immunogenically effective amount of one or more adenovirus 26 (Ad26) vectors together encoding four HIV antigens having the amino acid sequences of SEQ ID NO: 1, SEQ ID NO: 12, SEQ ID NO: 3, and SEQ ID NO: 4, and a pharmaceutically acceptable carrier; and
(ii) a booster vaccine comprising:
   (ii,a) a first booster vaccine composition comprising an immunogenically effective amount of one or more Ad26 vectors together encoding four HIV antigens having the amino acid sequences of SEQ ID NO: 1, SEQ ID NO: 12, SEQ ID NO: 3, and SEQ ID NO: 4, and a pharmaceutically acceptable carrier; and
   (ii, b) a second booster vaccine composition comprising at least one isolated HIV gp140 envelope polypeptide having the amino acid sequences of SEQ ID NO: 9 or SEQ ID NO: 10, preferably an isolated HIV gp140 polypeptide having the amino acid sequence of SEQ ID NO: 9 and an isolated HIV gp140 polypeptide having the amino acid sequence of SEQ ID NO: 10, an aluminum phosphate adjuvant and a pharmaceutically acceptable carrier.

The following examples of the invention are to further illustrate the nature of the invention. It should be understood that the following examples do not limit the invention and the scope of the invention is to be determined by the appended claims.

### EXAMPLES

### Example 1

### Study of HIV Vaccine Regimens in HIV-Infected Humans Undergoing Antiretroviral Therapy (ART)

Clinical studies in humans are conducted to investigate the effect of Ad26 vector priming immunizations and boosting immunizations of either MVA vectors or Ad26 vectors in combination with isolated HIV gp140 polypeptide in HIV-infected human adults undergoing anti-retroviral therapy (ART).

Objectives

The primary objective of the study is to determine the safety and tolerability of an Ad26 prime/MVA boost vaccine regimen and an Ad26 primer/Ad26 plus gp140 protein boost vaccine versus placebo in subjects on suppressive ART that was initiated outside of the acute phase of HIV infection. The secondary objectives of the study include: (1) determining the immunogenicity of the two different vaccine regimens in subjects on suppressive ART that was initiated outside of the acute phase of HIV infection; and (2) assessing the frequency, magnitude, specificity and functional capacity of humoral and cellular immune responses to the two different vaccine regimens.

Vaccination and Experimental Design

A single-center, randomized, parallel-group, placebo-controlled, double-blind Phase 1 clinical study in HIV-infected adults aged 18 to 55 years is performed. A target of 26 human subjects are participating in this study. Each subject has documented HIV-1 infection as confirmed by screening using a US FDA approved assay for diagnosing HIV infection. The subjects enrolled in the study started on antiretroviral therapy (ART) outside of the acute phase of HIV infection, i.e., not prior to entering the Fiebig VI phase of HIV infection. Each subject is on suppressive ART for at least 48 weeks as well as on stable suppressive ART for at least 24 weeks prior to initiation of vaccine/placebo, and has achieved absence of viremia (plasma HIV RNA of less than 50 copies/ml) for at least 24 weeks prior to initiation of vaccine/placebo. The subjects are divided into two groups: two test groups (10 subjects each) and the control group (6 subjects). The subjects in the test groups receive the study vaccine, and the subjects in control group receive placebo. The study continues for 36 weeks.

Dosage and Administration

Subjects receive four doses of study vaccine: adenovirus 26 vectors encoding mosaic HIV antigens (Ad26ₘₒₛ) or placebo is administered at weeks 0 and 12; and either (i) MVA vectors encoding mosaic HIV antigens (MVAₘₒₛ) (or placebo), or (ii) Ad26ₘₒₛ in combination with a mixture of HIV gp140 polypeptides (or placebo) is administered at Weeks 24 and 36. Study vaccines (Ad26ₘₒₛ and MVAₘₒₛ) and placebo with the administered doses are as follows:
(i) Ad26ₘₒₛ is composed of the following four vaccine products supplied pre-mixed in the same vial and administered in a 1:1:1:1 ratio of vps: Ad26.Mos1Env, Ad26.Mos2SEnv, Ad26.MoslGag-Pol, and Ad26.Mos2Gag-Pol expressing HIV mosaic Env1 (SEQ ID NO: 1), mosaic Env2S (SEQ ID NO: 12), mosaic GagPol1 (SEQ ID: NO 3), and mosaic GagPol2 (SEQ ID NO: 4) genes, respectively; total dose is about 5×10¹⁰ viral particles (vp) per 0.5 ml injection;
(ii) MVAₘₒₛ is composed of the following two vaccine products supplied in separate vials and administered in a 1:1 ratio: MVA-Mosaic1 (MVA virus expressing Mosaic1 HIV-1 Gag, Pol, and Env proteins having SEQ ID NOs: 1 and 3) and MVA-Mosaic2 (MVA virus expressing Mosaic2 HIV-1 Gag, Pol, and Env proteins having SEQ ID NOs: 2 and 4); total dose is about 1×10⁸ plaque forming units (pfu) per 0.5 ml injection;
(iii) Clade C gp140 (SEQ ID NO: 9) is a trimeric recombinant HIV-1 gp140 envelope protein of Clade C;
(iv) Mosaic gp140 (SEQ ID NO: 10) is a trimeric recombinant HIV-1 gp140 envelope protein engineered to contain motifs of multiple HIV-1 variants; and
(v) Placebo is 0.9% sodium chloride (0.5 ml injection).
   Clade C gp140 and mosaic gp140 are supplied in separate vials at a concentration of 1 mg/mL each. Prior to injection, mosaic gp140 is pre-mixed with clade C gp140 at a 1:1 (v/v) ratio. Then, aluminum phosphate (1.7 mg/mL) is mixed with the protein mixture at a 1:1 (v/v) ratio prior to injection. The total dose administered is about 125 μg clade C gp140, about 125 μg mosaic gp140, and about 425 μg aluminum per 0.5 ml injection.

Subjects receive the study vaccines or placebo according to the schedule in Table 1 below in four doses administered by intramuscular injection

TABLE 1
Schedule for administration of study vaccines

| Group | N | Week 0 | Week 12 | Week 24 | Week 36 |
|---|---|---|---|---|---|
| Test | 10 | Ad26ₘₒₛ | Ad26ₘₒₛ | MVAₘₒₛ + | MVAₘₒₛ + |
| Group 1 | | | | placebo | placebo |
| Test | 10 | Ad26ₘₒₛ | Ad26ₘₒₛ | Ad26ₘₒₛ + | Ad26ₘₒₛ + |
| Group 2 | | | | Clade C | Clade C |
| | | | | gp140 + | gp140 + |
| | | | | mosaic gp140 | mosaic gp140 |
| Control | 6 | Placebo | Placebo | Placebo | Placebo |

Subjects in both the test and control groups continue to receive standard ART (e.g. at least three antiviral compounds, e.g. two nucleoside reverse transcriptase inhibitors plus either non-nucleoside reverse transcriptase inhibitor or protease inhibitor or integrase inhibitor) for HIV treatment during the study. Blood and optionally genital secretions are taken at specific clinical visits to assess immune responses (cellular and humoral immune responses) and viremic control throughout the study.

It is understood that the examples and embodiments described herein are for illustrative purposes only, and that changes could be made to the embodiments described above without departing from the broad inventive concept thereof. It is understood, therefore, that this invention is not limited to the particular embodiments disclosed, but it is intended to cover modifications within the spirit and scope of the invention as defined by the appended claims.

SEQUENCE LISTING

SEQ ID NO: 1 (Mos1.Env) 685 aa:
MRVTGIRKNYQHLWRWGTMLLGILMICSAAGKLWVTVYYGVPVWKEATTTLFCASDAKAYDTEVHNVWATHACVPTDPNPQEVVLENVTEN
FNMWKNNMVEQMHEDIISLWDQSLKPCVKLTPLCVTLNCTDDVRNVTNNATNTNSSWGEPMEKGEIKNCSFNITTSIRNKVQKQYALFYKL
DVVPIDNDSNNTNYRLISCNTSVITQACPKVSFEPIPIHYCAPAGFAILKCNDKKFNGTGPCTNVSTVQCTHGIRPVVSTQLLLNGSLAEE
EVVIRSENFTNNAKTIMVQLNVSVEINCTRPNNNTRKSIHIGPGRAFYTAGDIIGDIRQAHCNISRANWNNTLRQIV
EKLGKQFGNNKTIVFNHSSGGDPEIVMHSFNCGGEFFYCNSTKLFNSTWTWNNSTWNNTKRSNDTEEHITLPCRIKQIINMWQEVGKAMYA
PPIRGQIRCSSNITGLLLTRDGGNDTSGTEIFRPGGGDMRDNWRSELYKYKVVKIEPLGVAPTKAKRRVVQSEKSAVGIGAVFLGFLGAAG
STMGAASMTLTVQARLLLSGIVQQQNNLLRAIEAQQHLLQLTVWGIKQLQARVLAVERYLKDQQLLGIWGCSGKLICTTTVPWNASWSNKS
LDKIWNNMTWMEWEREINNYTSLIYTLIEESQNQQEKNEQELLELDKWASLWNWFDISNWLW
SEQ ID NO: 2 (Mos2.Env) 684 aa:
MRVRGIQRNWPQWWIWGILGFWMIIICRVMGNLWVTVYYGVPVWKEAKTTLFCASDAKAYEKEVHNVWATHACVPTDPNPQEMVLENVTEN
FNMWKNDMVDQMHEDIIRLWDQSLKPCVKLTPLCVTLECRNVRNVSSNGTYNIIHNETYKEMKNCSFNATTVVEDRKQKVHALFYRLDIVP
LDENNSSEKSSENSSEYYRLINCNTSAITQACPKVSFDPIPIHYCAPAGYAILKCNNKTFNGTGPCNNVSTVQCTHGIKPVVSTQLLLNGS
LAEEEIIIRSENLTNNAKTIIVHLNETVNITCTRPNNNTRKSIRIGPGQTFYATGDIIGDIRQAHCNLSRDGWNKTLQGVKKKLAEHFPNK
TINFTSSSGGDLEITTHSFNCRGEFFYCNTSGLFNGTYMPNGTNSNSSSNITLPCRIKQIINMWQEVGRAMYAPPIAGNITCRSNITGLLL
TRDGGSNNGVPNDTETFRPGGGDMRNNWRSELYKYKVVEVKPLGVAPTEAKRRVVESEKSAVGIGAVFLGILGAAGSTMGAASITLTVQAR
QLLSGIVQQQSNLLRAIEAQQHMLQLTVWGIKQLQTRVLAIERYLQDQQLLGLWGCSGKLICTTAVPWNTSWSNKSQTDIWDNMTWMQWDK
EIGNYTGEIYRLLEESQNQQEKNEKDLLALDSWKNLWNWFDITNWLW
SEQ ID NO: 3 (Mos1.Gag-Pol) 1350 aa:
MGARASVLSGGELDRWEKIRLRPGGKKKYRLKHIVWASRELERFAVNPGLLETSEGCRQILGQLQPSLQTGSEELRSLYNTVATLYCVHQR
IEIKDTKEALEKIEEEQNKSKKKAQQAAADTGNSSQVSQNYPIVQNIQGQMVHQAISPRTLNAWVKVVEEKAFSPEVIPMFSALSEGATPQ
DLNTMLNTVGGHQAAMQMLKETINEEAAEWDRVHPVHAGPIAPGQMREPRGSDIAGTTSTLQEQIGWMTNNPPIPVGEIYKRWIILGLNKI
VRMYSPVSILDIRQGPKEPFRDYVDRFYKTLRAEQASQDVKNWMTETLLVQNANPDCKTILKALGPAATLEEMMTACQGVGGPGHKARVLA
EAMSQVTNSATIMMQRGNFRNQRKTVKCFNCGKEGHIAKNCRAPRKKGCWKCGKEGHQMKDCTERQANFLGKIWPSNKGRPGNFLQNRPEP
TAPPEESFRFGEETTTPSQKQEPIDKEMYPLASLKSLFGNDPSSQMAPISPIETVPVKLKPGMDGPRVKQWPLTEEKIKALTAICEEMEKE
GKITKIGPENPYNTPVFAIKKKDSTKWRKLVDFRELNKRTQDFWEVQLGIPHPAGLKKKKSVTVLAVGDAYFSVPLDEGFRKYTAFTIPST
NNETPGIRYQYNVLPQGWKGSPAIFQCSMTRILEPFRAKNPEIVIYQYMAALYVGSDLEIGQHRAKIEELREHLLKWGFTTPDKKHQKEPP
FLWMGYELHPDKWTVQPIQLPEKDSWTVNDIQKLVGKLNWASQIYPGIKVRQLCKLLRGAKALTDIVPLTEEAELELAENREILKEPVHGV
YYDPSKDLIAEIQKQGHDQWTYQIYQEPFKNLKTGKYAKMRTAHTNDVKQLTEAVQKIAMESIVIWGKTPKFRLPIQKETWETWWTDYWQA
TWIPEWEFVNTPPLVKLWYQLEKDPIAGVETFYVAGAANRETKLGKAGYVTDRGRQKIVSLTETTNQKTALQAIYLALQDSGSEVNIVTAS
QYALGIIQAQPDKSESELVNQIIEQLIKKERVYLSWVPAHKGIGGNEQVDKLVSSGIRKVLFLDGIDKAQEEHEKYHSNWRAMASDFNLPP
VVAKEIVASCDQCQLKGEAMHGQVDCSPGIWQLACTHLEGKIILVAVHVASGYIEAEVIPAETGQETAYFILKLAGRWPVKVIHTANGSNF
TSAAVKAACWWAGIQQEFGIPYNPQSQGVVASMNKELKKIIGQVRDQAEHLKTAVQMAVFIHNFKRKGGIGGYSAGERIIDIIATDIQTKE
LQKQIIKIQNFRVYYRDSRDPIWKGPAKLLWKGEGAVVIQDNSDIKVVPRRKVKIIKDYGKQMAGADCVAGRQDED
SEQ ID NO: 4 (Mos2.Gag-Pol) 1341 aa:
MGARASILRGGKLDKWEKIRLRPGGKKHYMLKHLVWASRELERFALNPGLLETSEGCKQIIKQLQPALQTGTEELRSLFNTVATLYCVHAE
IEVRDTKEALDKIEEEQNKSQQKTQQAKEADGKVSQNYPIVQNLQGQMVHQPISPRTLNAWVKVIEEKAFSPEVIPMFTALSEGATPQDLN
TMLNTVGGHQAAMQMLKDTINEEAAEWDRLHPVHAGPVAPGQMREPRGSDIAGTTSNLQEQIAWMTSNPPIPVGDIYKRWIILGLNKIVRM
YSPTSILDIKQGPKEPFRDYVDRFFKTLRAEQATQDVKNWMTDTLLVQNANPDCKTILRALGPGATLEEMMTACQGVGGPSHKARVLAEAM
SQTNSTILMQRSNFKGSKRIVKCFNCGKEGHIARNCRAPRKKGCWKCGKEGHQMKDCTERQANFLGKIWPSHKGRPGNFLQSRPEPTAPPA
ESFRFEETTPAPKQEPKDREPLTSLRSLFGSDPLSQMAPISPIETVPVKLKPGMDGPKVKQWPLTEEKIKALVEICTEMEKEGKISKIGPE
NPYNTPIFAIKKKDSTKWRKLVDFRELNKRTQDFWEVQLGIPHPAGLKKKKSVTVLAVGDAYFSVPLDEDFRKYTAFTIPSINNETPGIRY
QYNVLPQGWKGSPAIFQSSMTKILEPFRKQNPDIVIYQYMAALYVGSDLEIGQHRTKIEELRQHLLRWGFTTPDKKHQKEPPFLWMGYELH
PDKWTVQPIVLPEKDSWTVNDIQKLVGKLNWASQIYAGIKVKQLCKLLRGTKALTEVVPLTEEAELELAENREILKEPVHGVYYDPSKDLI
AEIQKQGQGQWTYQIYQEPFKNLKTGKYARMRGAHTNDVKQLTEAVQKIATESIVIWGKTPKFKLPIQKETWEAWWTEYWQATWIPEWEFV
NTPPLVKLWYQLEKEPIVGAETFYVAGAANRETKLGKAGYVTDRGRQKVVSLTDTTNQKTALQAIHLALQDSGLEVNIVTASQYALGIIQA
QPDKSESELVSQIIEQLIKKEKVYLAWVPAHKGIGGNEQVDKLVSRGIRKVLFLDGIDKAQEEHEKYHSNWRAMASEFNLPPIVAKEIVAS
CDKCQLKGEAIHGQVDCSPGIWQLACTHLEGKVILVAVHVASGYIEAEVIPAETGQETAYFLLKLAGRWPVKTIHTANGSNFTSATVKAAC
WWAGIKQEFGIPYNPQSQGVVASINKELKKIIGQVRDQAEHLKTAVQMAVFIHNFKRKGGIGEYSAGERIVDIIASDIQTKELQKQITKIQ
NFRVYYRDSRDPLWKGPAKLLWKGEGAVVIQDNSDIKVVPRRKAKIIRDYGKQMAGDDCVASRQDED
SEQ ID NO: 5 (Mos1.Env DNA)
ATGCGGGTCACCGGCATCCGGAAGAACTACCAGCACCTGTGGCGGTGGGGCACCATGCTGCTGGGCATCCTGATGATTTGCTCTGCCGCCG
GAAAGCTGTGGGTGACCGTGTACTACGGCGTGCCCGTGTGGAAAGAGGCCACCACCACCCTGTTCTGCGCCAGCGACGCCAAGGCCTACGA
CACCGAGGTGCACAACGTGTGGGCCACCCACGCCTGCGTGCCCACCGACCCCAACCCCCAGGAAGTGGTCCTGGAAAACGTGACCGAGAAC
TTCAACATGTGGAAGAACAACATGGTGGAGCAGATGCACGAGGACATCATCAGCCTGTGGGACCAGAGCCTGAAGCCCTGCGTGAAGCTGA
CCCCCCTGTGCGTGACCCTGAACTGCACCGACGACGTGCGGAACGTGACCAACAACGCCACCAACACCAACAGCAGCTGGGGCGAGCCTAT
GGAAAAGGGCGAGATCAAGAACTGCAGCTTCAACATCACCACCTCCATCCGGAACAAGGTGCAGAAGCAGTACGCCCTGTTCTACAAGCTG
GACGTGGTGCCCATCGACAACGACAGCAACAACACCAACTACCGGCTGATCAGCTGCAACACCAGCGTGATCACCCAGGCCTGCCCCAAGG
TGTCCTTCGAGCCCATCCCCATCCACTACTGCGCCCCTGCCGGCTTCGCCATCCTGAAGTGCAACGACAAGAAGTTCAACGGCACCGGCCC
CTGCACCAACGTGAGCACCGTGCAGTGCACCCACGGCATCCGGCCCGTGGTGTCCACCCAGCTGCTGCTGAACGGCAGCCTGGCCGAGGAA
GAGGTGGTGATCAGAAGCGAGAATTTCACCAACAATGCCAAGACCATCATGGTGCAGCTGAACGTGAGCGTGGAGATCAACTGCACCCGGC
CCAACAACAACACCCGGAAGAGCATCCACATCGGCCCTGGCAGGGCCTTCTACACAGCCGGCGACATCATCGGCGACATCCGGCAGGCCCA
CTGCAACATCAGCCGGGCCAACTGGAACAACACCCTGCGGCAGATCGTGGAGAAGCTGGGCAAGCAGTTCGGCAACAACAAGACCATCGTG
TTCAACCACAGCAGCGGCGGAGACCCCGAGATCGTGATGCACAGCTTCAACTGTGGCGGCGAGTTCTTCTACTGCAACAGCACCAAGCTGT
TCAACAGCACCTGGACCTGGAACAACTCCACCTGGAATAACACCAAGCGGAGCAACGACACCGAAGAGCACATCACCCTGCCCTGCCGGAT
CAAGCAGATTATCAATATGTGGCAGGAGGTCGGCAAGGCCATGTACGCCCCTCCCATCCGGGGCCAGATCCGGTGCAGCAGCAACATCACC
GGCCTGCTGCTGACCCGGGACGGCGGCAACGATACCAGCGGCACCGAGATCTTCCGGCCTGGCGGCGGAGATATGCGGGACAACTGGCGGA
GCGAGCTGTACAAGTACAAGGTGGTGAAGATCGAGCCCCTGGGCGTGGCTCCCACCAAGGCCAAGCGGCGGGTGGTGCAGAGCGAGAAGAG
CGCCGTGGGCATCGGCGCCGTGTTTCTGGGCTTCCTGGGAGCCGCCGGAAGCACCATGGGAGCCGCCAGCATGACCCTGACCGTGCAGGCC
CGGCTGCTGCTGTCCGGCATCGTGCAGCAGCAGAACAACCTGCTCCGGGCCATCGAGGCCCAGCAGCACCTGCTGCAGCTGACCGTGTGGG
GCATCAAGCAGCTGCAGGCCAGGGTGCTGGCCGTGGAGAGATACCTGAAGGATCAGCAGCTCCTGGGGATCTGGGGCTGCAGCGGCAAGCT
GATCTGCACCACCACCGTGCCCTGGAACGCCAGCTGGTCCAACAAGAGCCTGGACAAGATCTGGAACAATATGACCTGGATGGAATGGGAG
CGCGAGATCAACAATTACACCAGCCTGATCTACACCCTGATCGAGGAAAGCCAGAACCAGCAGGAAAAGAACGAGCAGGAACTGCTGGAAC
TGGACAAGTGGGCCAGCCTGTGGAACTGGTTCGACATCAGCAACTGGCTGTGG
SEQ ID NO: 6 (Mos2.Env DNA)
ATGAGAGTGCGGGGCATCCAGCGGAACTGGCCCCAGTGGTGGATCTGGGGCATCCTGGGCTTTTGGATGATCATCATCTGCCGGGTGATGG
GCAACCTGTGGGTGACCGTGTACTACGGCGTGCCCGTGTGGAAAGAGGCCAAGACCACCCTGTTCTGCGCCAGCGACGCCAAGGCCTACGA
GAAAGAGGTGCACAACGTGTGGGCCACCCACGCCTGCGTGCCCACCGACCCCAACCCCCAGGAAATGGTCCTGGAAAACGTGACCGAGAAC
TTCAACATGTGGAAGAACGACATGGTGGACCAGATGCACGAGGACATCATCCGGCTGTGGGACCAGAGCCTGAAGCCCTGCGTGAAGCTGA
CCCCCCTGTGCGTGACCCTGGAATGCCGGAACGTGAGAAACGTGAGCAGCAACGGCACCTACAACATCATCCACAACGAGACCTACAAAGA
GATGAAGAACTGCAGCTTCAACGCCACCACCGTGGTGGAGGACCGGAAGCAGAAGGTGCACGCCCTGTTCTACCGGCTGGACATCGTGCCC
CTGGACGAGAACAACAGCAGCGAGAAGTCCAGCGAGAACAGCTCCGAGTACTACCGGCTGATCAACTGCAACACCAGCGCCATCACCCAGG
CCTGCCCCAAGGTGTCCTTCGACCCCATCCCCATCCACTACTGCGCCCCTGCCGGCTACGCCATCCTGAAGTGCAACAACAAGACCTTCAA
CGGCACCGGCCCCTGCAACAACGTGAGCACCGTGCAGTGCACCCACGGCATCAAGCCCGTGGTGTCCACCCAGCTGCTGCTGAACGGCAGC
CTGGCCGAGGAAGAGATCATCATCCGGTCCGAGAACCTGACCAACAACGCCAAGACCATCATCGTGCACCTGAATGAGACCGTGAACATCA
CCTGCACCCGGCCCAACAACAACACCCGGAAGAGCATCCGGATCGGCCCTGGCCAGACCTTTTACGCCACCGGCGACATCATCGGCGACAT
CCGGCAGGCCCACTGCAACCTGAGCCGGGACGGCTGGAACAAGACCCTGCAGGGCGTGAAGAAGAAGCTGGCCGAGCACTTCCCCAATAAG
ACCATCAACTTCACCAGCAGCAGCGGCGGAGACCTGGAAATCACCACCCACAGCTTCAACTGCAGGGGCGAGTTCTTCTACTGCAATACCT
CCGGCCTGTTCAATGGCACCTACATGCCCAACGGCACCAACAGCAACAGCAGCAGCAACATCACCCTGCCCTGCCGGATCAAGCAGATCAT
CAATATGTGGCAGGAGGTCGGCAGGGCCATGTACGCCCCTCCCATCGCCGGCAATATCACCTGCCGGTCCAACATCACCGGCCTGCTGCTG
ACCAGGGACGGCGGCAGCAACAACGGCGTGCCTAACGACACCGAGACCTTCCGGCCTGGCGGCGGAGATATGCGGAACAACTGGCGGAGCG
AGCTGTACAAGTACAAGGTGGTGGAGGTGAAGCCCCTGGGCGTGGCTCCTACCGAGGCCAAGCGGCGGGTGGTGGAGAGCGAGAAGAGCGC
CGTGGGCATCGGCGCCGTGTTTCTGGGCATTCTGGGAGCCGCCGGAAGCACCATGGGAGCCGCCAGCATCACCCTGACCGTGCAGGCCCGG
CAGCTGCTGTCCGGCATCGTGCAGCAGCAGAGCAACCTGCTGAGAGCCATCGAGGCCCAGCAGCACATGCTGCAGCTCACCGTGTGGGGCA
TCAAGCAGCTGCAGACCCGGGTGCTGGCCATCGAGAGATACCTGCAGGATCAGCAGCTCCTGGGCCTGTGGGGCTGCAGCGGCAAGCTGAT
CTGCACCACCGCCGTGCCCTGGAACACCAGCTGGTCCAACAAGAGCCAGACCGACATCTGGGACAACATGACCTGGATGCAGTGGGACAAA
GAGATCGGCAACTACACCGGCGAGATCTACAGGCTGCTGGAAGAGAGCCAGAACCAGCAGGAAAAGAACGAGAAGGACCTGCTGGCCCTGG
ACAGCTGGAAGAACCTGTGGAACTGGTTCGACATCACCAACTGGCTGTGG
SEQ ID NO: 7 (Mos1.Gag-Pol DNA)
ATGGGAGCCAGAGCCAGCGTGCTGTCCGGAGGGGAGCTGGACCGCTGGGAGAAGATCAGGCTGAGGCCTGGAGGGAAGAAGAAGTACAGGC
TGAAGCACATCGTGTGGGCCAGCAGAGAGCTGGAACGGTTTGCCGTGAACCCTGGCCTGCTGGAAACCAGCGAGGGCTGTAGGCAGATTCT
GGGACAGCTGCAGCCCAGCCTGCAGACAGGCAGCGAGGAACTGCGGAGCCTGTACAACACCGTGGCCACCCTGTACTGCGTGCACCAGCGG
ATCGAGATCAAGGACACCAAAGAAGCCCTGGAAAAGATCGAGGAAGAGCAGAACAAGAGCAAGAAGAAAGCCCAGCAGGCTGCCGCTGACA
CAGGCAACAGCAGCCAGGTGTCCCAGAACTACCCCATCGTGCAGAACATCCAGGGACAGATGGTGCACCAGGCCATCAGCCCTCGGACCCT
GAACGCCTGGGTGAAGGTGGTGGAGGAAAAGGCCTTCAGCCCTGAGGTGATCCCCATGTTCTCTGCCCTGAGCGAGGGAGCCACACCCCAG
GACCTGAACACCATGCTGAACACCGTGGGAGGGCACCAGGCTGCCATGCAGATGCTGAAAGAGACAATCAACGAGGAAGCTGCCGAGTGGG
ACAGGGTCCACCCAGTGCACGCTGGACCTATCGCTCCTGGCCAGATGAGAGAGCCCAGAGGCAGCGATATTGCTGGCACCACCTCCACACT
GCAGGAACAGATCGGCTGGATGACCAACAACCCTCCCATCCCTGTGGGAGAGATCTACAAGCGGTGGATCATTCTGGGACTGAACAAGATC
GTGCGGATGTACAGCCCTGTGAGCATCCTGGACATCAGGCAGGGACCCAAAGAGCCCTTCAGGGACTACGTGGACCGGTTCTACAAGACCC
TGAGAGCCGAGCAGGCCAGCCAGGACGTGAAGAACTGGATGACCGAGACACTGCTGGTGCAGAACGCCAACCCTGACTGCAAGACCATCCT
GAAAGCCCTGGGACCTGCTGCCACCCTGGAAGAGATGATGACAGCCTGCCAGGGAGTGGGAGGACCTGGCCACAAGGCCAGGGTGCTGGCC
GAGGCCATGAGCCAGGTGACCAACTCTGCCACCATCATGATGCAGAGAGGCAACTTCCGGAACCAGAGAAAGACCGTGAAGTGCTTCAACT
GTGGCAAAGAGGGACACATTGCCAAGAACTGCAGGGCTCCCAGGAAGAAAGGCTGCTGGAAGTGCGGAAAAGAAGGCCACCAGATGAAGGA
CTGCACCGAGAGGCAGGCCAACTTCCTGGGCAAGATCTGGCCTAGCAACAAGGGCAGGCCTGGCAACTTCCTGCAGAACAGACCCGAGCCC
ACCGCTCCTCCCGAGGAAAGCTTCCGGTTTGGCGAGGAAACCACCACCCCTAGCCAGAAGCAGGAACCCATCGACAAAGAGATGTACCCTC
TGGCCAGCCTGAAGAGCCTGTTCGGCAACGACCCCAGCAGCCAGATGGCTCCCATCAGCCCAATCGAGACAGTGCCTGTGAAGCTGAAGCC
TGGCATGGACGGACCCAGGGTGAAGCAGTGGCCTCTGACCGAGGAAAAGATCAAAGCCCTGACAGCCATCTGCGAGGAAATGGAAAAAGAG
GGCAAGATCACCAAGATCGGACCCGAGAACCCCTACAACACCCCTGTGTTCGCCATCAAGAAGAAAGACAGCACCAAGTGGAGGAAACTGG
TGGACTTCAGAGAGCTGAACAAGCGGACCCAGGACTTCTGGGAGGTGCAGCTGGGCATCCCTCACCCTGCTGGCCTGAAGAAAAAGAAAAG
CGTGACCGTGCTGGCTGTGGGAGATGCCTACTTCAGCGTGCCTCTGGACGAGGGCTTCCGGAAGTACACAGCCTTCACCATCCCCAGCACC
AACAACGAGACACCTGGCATCAGATACCAGTACAACGTGCTGCCTCAGGGCTGGAAAGGCAGCCCTGCCATCTTCCAGTGCAGCATGACCA
GAATCCTGGAACCCTTCAGAGCCAAGAACCCTGAGATCGTGATCTACCAGTATATGGCTGCCCTCTACGTGGGCAGCGACCTGGAAATCGG
ACAGCACAGAGCCAAAATCGAAGAACTCCGCGAGCACCTGCTGAAGTGGGGATTCACCACCCCTGACAAGAAGCACCAGAAAGAGCCTCCC
TTCCTGTGGATGGGCTACGAGCTGCACCCTGACAAGTGGACCGTGCAGCCCATCCAGCTGCCAGAGAAGGACTCCTGGACCGTGAACGACA
TCCAGAAACTGGTCGGCAAGCTGAACTGGGCCAGCCAGATCTACCCTGGCATCAAAGTCAGACAGCTGTGTAAGCTGCTGAGGGGAGCCAA
AGCACTGACCGACATCGTGCCTCTGACAGAAGAAGCCGAGCTGGAACTGGCCGAGAACAGAGAGATCCTGAAAGAACCCGTGCACGGAGTG
TACTACGACCCCTCCAAGGACCTGATTGCCGAGATCCAGAAACAGGGACACGACCAGTGGACCTACCAGATCTATCAGGAACCTTTCAAGA
ACCTGAAAACAGGCAAGTACGCCAAGATGCGGACAGCCCACACCAACGACGTGAAGCAGCTGACCGAAGCCGTGCAGAAAATCGCCATGGA
AAGCATCGTGATCTGGGGAAAGACACCCAAGTTCAGGCTGCCCATCCAGAAAGAGACATGGGAAACCTGGTGGACCGACTACTGGCAGGCC
ACCTGGATTCCCGAGTGGGAGTTCGTGAACACCCCACCCCTGGTGAAGCTGTGGTATCAGCTGGAAAAGGACCCTATCGCTGGCGTGGAGA
CATTCTACGTGGCTGGAGCTGCCAACAGAGAGACAAAGCTGGGCAAGGCTGGCTACGTGACCGACAGAGGCAGACAGAAAATCGTGAGCCT
GACCGAAACCACCAACCAGAAAACAGCCCTGCAGGCCATCTATCTGGCACTGCAGGACAGCGGAAGCGAGGTGAACATCGTGACAGCCAGC
CAGTATGCCCTGGGCATCATCCAGGCCCAGCCTGACAAGAGCGAGAGCGAGCTGGTGAACCAGATCATCGAGCAGCTGATCAAGAAAGAAC
GGGTGTACCTGAGCTGGGTGCCAGCCCACAAGGGCATCGGAGGGAACGAGCAGGTGGACAAGCTGGTGTCCAGCGGAATCCGGAAGGTGCT
GTTCCTGGACGGCATCGATAAAGCCCAGGAAGAGCACGAGAAGTACCACAGCAATTGGAGAGCCATGGCCAGCGACTTCAACCTGCCTCCC
GTGGTGGCCAAAGAAATCGTGGCCAGCTGCGACCAGTGCCAGCTGAAAGGCGAGGCCATGCACGGACAGGTGGACTGCTCCCCTGGCATCT
GGCAGCTGGCATGCACCCACCTGGAAGGCAAGATCATTCTGGTGGCCGTGCACGTGGCCAGCGGATACATCGAAGCCGAAGTGATCCCTGC
CGAGACAGGGCAGGAAACAGCCTACTTCATCCTGAAGCTGGCTGGCAGATGGCCTGTGAAGGTGATCCACACAGCCAACGGCAGCAACTTC
ACCTCTGCTGCCGTGAAGGCTGCCTGTTGGTGGGCTGGCATTCAGCAGGAATTTGGCATCCCCTACAATCCCCAGTCTCAGGGAGTGGTGG
CCAGCATGAACAAAGAGCTGAAGAAGATCATCGGACAGGTCAGGGATCAGGCCGAGCACCTGAAAACTGCCGTCCAGATGGCCGTGTTCAT
CCACAACTTCAAGCGGAAGGGAGGGATCGGAGGGTACTCTGCTGGCGAGCGGATCATCGACATCATTGCCACCGATATCCAGACCAAAGAG
CTGCAGAAACAGATCATCAAGATCCAGAACTTCAGGGTGTACTACAGGGACAGCAGGGACCCCATCTGGAAGGGACCTGCCAAGCTGCTGT
GGAAAGGCGAAGGAGCCGTCGTCATCCAGGACAACAGCGACATCAAGGTGGTGCCCAGACGGAAGGTGAAAATCATCAAGGACTACGGCAA
ACAGATGGCTGGAGCCGACTGTGTCGCTGGCAGGCAGGACGAGGAC
SEQ ID NO: 8 (Mos2.Gag-Pol DNA)
ATGGGAGCCAGAGCCAGCATCCTGCGAGGAGGGAAGCTGGACAAGTGGGAGAAGATCAGGCTGAGGCCTGGAGGGAAGAAACACTACATGC
TGAAGCACCTGGTCTGGGCCAGCAGAGAGCTGGAACGGTTTGCCCTCAATCCTGGCCTGCTGGAAACCAGCGAGGGCTGCAAGCAGATCAT
CAAGCAGCTGCAGCCTGCCCTGCAGACAGGCACCGAGGAACTGCGGAGCCTGTTCAACACCGTGGCCACCCTGTACTGCGTGCATGCCGAG
ATCGAAGTGAGGGACACCAAAGAAGCCCTGGACAAGATCGAGGAAGAGCAGAACAAGAGCCAGCAGAAAACCCAGCAGGCCAAAGAAGCCG
ACGGCAAGGTCTCCCAGAACTACCCCATCGTGCAGAACCTGCAGGGACAGATGGTGCACCAGCCCATCAGCCCTCGGACACTGAATGCCTG
GGTGAAGGTGATCGAGGAAAAGGCCTTCAGCCCTGAGGTGATCCCCATGTTCACAGCCCTGAGCGAGGGAGCCACACCCCAGGACCTGAAC
ACCATGCTGAACACCGTGGGAGGGCACCAGGCTGCCATGCAGATGCTGAAGGACACCATCAACGAGGAAGCTGCCGAGTGGGACAGGCTGC
ACCCTGTGCACGCTGGACCTGTGGCTCCTGGCCAGATGAGAGAGCCCAGAGGCAGCGATATTGCTGGCACCACCTCCAATCTGCAGGAACA
GATCGCCTGGATGACCAGCAACCCTCCCATCCCTGTGGGAGACATCTACAAGCGGTGGATCATCCTGGGACTGAACAAGATCGTGCGGATG
TACAGCCCTACCTCCATCCTGGACATCAAGCAGGGACCCAAAGAGCCTTTCAGGGACTACGTGGACCGGTTCTTCAAGACCCTGAGAGCCG
AGCAGGCCACCCAGGACGTGAAGAACTGGATGACCGACACCCTGCTGGTGCAGAACGCCAACCCTGACTGCAAGACCATCCTGAGAGCCCT
GGGACCTGGAGCCACCCTGGAAGAGATGATGACAGCCTGCCAGGGAGTGGGAGGACCCTCTCACAAGGCTAGGGTGCTGGCCGAGGCCATG
AGCCAGACCAACAGCACCATCCTGATGCAGCGGAGCAACTTCAAGGGCAGCAAGCGGATCGTGAAGTGCTTCAACTGTGGCAAAGAGGGAC
ACATTGCCAGAAACTGTAGGGCACCCAGGAAGAAAGGCTGCTGGAAGTGCGGAAAAGAAGGCCACCAGATGAAGGACTGCACCGAGAGGCA
GGCCAACTTCCTGGGCAAGATCTGGCCTAGCCACAAGGGCAGACCTGGCAACTTCCTGCAGAGCAGACCCGAGCCCACCGCTCCTCCAGCC
GAGAGCTTCCGGTTCGAGGAAACCACCCCTGCTCCCAAGCAGGAACCTAAGGACAGAGAGCCTCTGACCAGCCTGAGAAGCCTGTTCGGCA
GCGACCCTCTGAGCCAGATGGCTCCCATCTCCCCTATCGAGACAGTGCCTGTGAAGCTGAAGCCTGGCATGGACGGACCCAAGGTGAAACA
GTGGCCTCTGACCGAGGAAAAGATCAAAGCCCTGGTGGAGATCTGTACCGAGATGGAAAAAGAGGGCAAGATCAGCAAGATCGGACCCGAG
AACCCCTACAACACCCCTATCTTCGCCATCAAGAAGAAAGACAGCACCAAGTGGAGGAAACTGGTGGACTTCAGAGAGCTGAACAAGCGGA
CCCAGGACTTCTGGGAGGTGCAGCTGGGCATCCCTCACCCTGCTGGCCTGAAGAAAAAGAAAAGCGTGACCGTGCTGGCCGTGGGAGATGC
CTACTTCAGCGTGCCTCTGGACGAGGACTTCAGAAAGTACACAGCCTTCACCATCCCCAGCATCAACAACGAGACACCTGGCATCAGATAC
CAGTACAACGTGCTGCCTCAGGGATGGAAGGGCTCTCCTGCAATCTTCCAGAGCAGCATGACCAAGATCCTGGAACCCTTCCGGAAGCAGA
ACCCTGACATCGTGATCTACCAGTACATGGCAGCCCTGTACGTCGGCAGCGACCTGGAAATCGGACAGCACCGGACCAAGATCGAAGAACT
CAGGCAGCACCTGCTGCGGTGGGGATTCACCACCCCTGACAAGAAGCACCAGAAAGAGCCTCCCTTCCTGTGGATGGGCTACGAGCTGCAC
CCAGACAAGTGGACCGTGCAGCCCATCGTGCTGCCTGAGAAGGACTCCTGGACCGTGAACGACATCCAGAAACTGGTCGGCAAGCTGAACT
GGGCCAGCCAGATCTACGCTGGCATCAAAGTGAAGCAGCTGTGTAAGCTCCTGAGAGGCACCAAAGCCCTGACCGAGGTGGTGCCACTGAC
AGAGGAAGCCGAGCTGGAACTGGCCGAGAACAGAGAGATCCTGAAAGAACCCGTGCACGGAGTGTACTACGACCCCAGCAAGGACCTGATT
GCCGAGATCCAGAAGCAGGGACAGGGACAGTGGACCTACCAGATCTACCAGGAACCCTTCAAGAACCTGAAAACAGGCAAGTACGCCAGGA
TGAGGGGAGCCCACACCAACGACGTCAAACAGCTGACCGAAGCCGTGCAGAAGATCGCCACCGAGAGCATCGTGATTTGGGGAAAGACACC
CAAGTTCAAGCTGCCCATCCAGAAAGAGACATGGGAGGCCTGGTGGACCGAGTACTGGCAGGCCACCTGGATTCCCGAGTGGGAGTTCGTG
AACACCCCACCCCTGGTGAAGCTGTGGTATCAGCTGGAAAAAGAACCCATCGTGGGAGCCGAGACATTCTACGTGGCTGGAGCTGCCAACA
GAGAGACAAAGCTGGGCAAGGCTGGCTACGTGACCGACAGAGGCAGGCAGAAAGTGGTGTCCCTGACCGATACCACCAACCAGAAAACAGC
CCTGCAGGCCATCCACCTGGCTCTGCAGGACTCTGGCCTGGAAGTGAACATCGTGACAGCCAGCCAGTATGCCCTGGGCATCATTCAGGCA
CAGCCTGACAAGAGCGAGAGCGAGCTGGTGTCTCAGATCATTGAGCAGCTGATCAAGAAAGAAAAGGTGTACCTGGCCTGGGTGCCAGCCC
ACAAGGGGATCGGAGGGAACGAGCAGGTGGACAAGCTGGTGTCCAGGGGCATCCGGAAGGTGCTGTTTCTGGACGGCATCGACAAAGCCCA
GGAAGAGCACGAGAAGTACCACAGCAATTGGAGAGCCATGGCCAGCGAGTTCAACCTGCCTCCCATCGTGGCCAAAGAAATCGTGGCCTCT
TGCGACAAGTGCCAGCTGAAAGGCGAGGCCATTCACGGACAGGTGGACTGCAGCCCAGGCATCTGGCAGCTGGCCTGCACCCACCTGGAAG
GCAAGGTGATCCTGGTGGCCGTGCACGTGGCCTCTGGATACATCGAAGCCGAAGTGATCCCTGCCGAGACAGGCCAGGAAACAGCCTACTT
CCTGCTGAAGCTGGCTGGCAGGTGGCCTGTGAAAACCATCCACACAGCCAACGGCAGCAACTTCACCTCTGCCACCGTGAAGGCTGCCTGT
TGGTGGGCTGGCATTAAGCAGGAATTTGGCATCCCCTACAACCCTCAGTCTCAGGGAGTGGTGGCCTCCATCAACAAAGAGCTGAAGAAGA
TCATCGGACAGGTCAGGGATCAGGCCGAGCATCTGAAAACAGCCGTCCAGATGGCCGTGTTCATCCACAACTTCAAGCGGAAGGGAGGGAT
CGGAGAGTACTCTGCTGGCGAGAGGATCGTGGACATTATCGCCAGCGATATCCAGACCAAAGAACTGCAGAAGCAGATCACAAAGATCCAG
AACTTCAGGGTGTACTACAGGGACAGCAGAGATCCCCTGTGGAAGGGACCTGCCAAGCTGCTGTGGAAAGGCGAAGGAGCCGTCGTCATCC
AGGACAACAGCGACATCAAGGTGGTGCCCAGACGGAAGGCCAAGATCATCAGAGACTACGGCAAACAGATGGCTGGCGACGACTGCGTCGC
CTCTAGGCAGGACGAGGAC
SEQ ID NO: 9 (Clade C gp140 protein)- 679 amino acids
AENLWVGNMW VTVYYGVPVW TDAKTTLFCA SDTKAYDREV HNVWATHACV PTDPNPQEIV
LENVTENFNM WKNDMVDQMH EDIISLWDQS LKPCVKLTPL CVTLHCTNAT FKNNVTNDMN
KEIRNCSFNT TTEIRDKKQQ GYALFYRPDI VLLKENRNNS NNSEYILINC NASTITQACP
KVNFDPIPIH YCAPAGYAIL KCNNKTFSGK GPCNNVSTVQ CTHGIKPVVS TQLLLNGSLA
EKEIIIRSEN LTDNVKTIIV HLNKSVEIVC TRPNNNTRKS MRIGPGQTFY ATGDIIGDIR
QAYCNISGSK WNETLKRVKE KLQENYNNNK TIKFAPSSGG DLEITTHSFN CRGEFFYCNT
TRLFNNNATE DETITLPCRI KQIINMWQGV GRAMYAPPIA GNITCKSNIT GLLLVRDGGE
DNKTEEIFRP GGGNMKDNWR SELYKYKVIE LKPLGIAPTG AKERVVEREE RAVGIGAVFL
GFLGAAGSTM GAASLTLTVQ ARQLLSSIVQ QQSNLLRAIE AQQHMLQLTV WGIKQLQTRV
LAIERYLKDQ QLLGIWGCSG KLICTTNVPW NSSWSNKSQT DIWNNMTWME WDREISNYTD
TIYRLLEDSQ TQQEKNEKDL LALDSWKNLW SWFDISNWLW YIKSRIEGRG SGGYIPEAPR
DGQAYVRKDG EWVLLSTFL
SEQ ID NO: 10 (Mosaic gp140 protein)
AGKLWVTVYY GVPVWKEATT TLFCASDAKA YDTEVHNVWA THACVPTDPN PQEVVLENVT
ENFNMWKNNM VEQMHEDIIS LWDQSLKPCV KLTPLCVTLN CTDDVRNVTN NATNTNSSWG
EPMEKGEIKN CSFNITTSIR NKVQKQYALF YKLDVVPIDN DSNNTNYRLI SCNTSVITQA
CPKVSFEPIP IHYCAPAGFA ILKCNDKKFN GTGPCTNVST VQCTHGIRPV VSTQLLLNGS
LAEEEVVIRS ENFTNNAKTI MVQLNVSVEI NCTRPNNNTR KSIHIGPGRA FYTAGDIIGD
IRQAHCNISR ANWNNTLRQI VEKLGKQFGN NKTIVFNHSS GGDPEIVMHS FNCGGEFFYC
NSTKLFNSTW TWNNSTWNNT KRSNDTEEHI TLPCRIKQII NMWQEVGKAM YAPPIRGQIR
CSSNITGLLL TRDGGNDTSG TEIFRPGGGD MRDNWRSELY KYKVVKIEPL GVAPTKAKER
VVQREERAVG IGAVFLGFLG AAGSTMGAAS MTLTVQARLL LSGIVQQQNN LLRAIEAQQH
LLQLTVWGIK QLQARVLAVE RYLKDQQLLG IWGCSGKLIC TTTVPWNASW SNKSLDKIWN
NMTWMEWERE INNYTSLIYT LIEESQNQQE KNEQELLELD KWASLWNWFD ISNWLWYIKS
RIEGRGSGGY IPEAPRDGQA YVRKDGEWVL LSTFL
SEQ ID NO: 11 (exemplary leader sequence for gp140 stabilized trimeric protein
production)
MRVRGIQRNC QHLWRWGTLI LGMLMICSA
SEQ ID NO: 12 (Mos2SEnv)
MRVRGMLRNW QQWWIWSSLG FWMLMIYSVM GNLWVTVYYG VPVWKDAKTT LFCASDAKAY
EKEVHNVWAT HACVPTDPNP QEIVLGNVTE NFNMWKNDMV DQMHEDIISL WDASLEPCVK
LTPLCVTLNC RNVRNVSSNG TYNIIHNETY KEMKNCSFNA TTVVEDRKQK VHALFYRLDI
VPLDENNSSE KSSENSSEYY RLINCNTSAI TQACPKVSFD PIPIHYCAPA GYAILKCNNK
TFNGTGPCNN VSTVQCTHGI KPVVSTQLLL NGSLAEEEII IRSENLTNNA KTIIVHLNET
VNITCTRPNN NTRKSIRIGP GQTFYATGDI IGDIRQAHCN LSRDGWNKTL QGVKKKLAEH
FPNKTIKFAP HSGGDLEITT HTFNCRGEFF YCNTSNLFNE SNIERNDSII TLPCRIKQII
NMWQEVGRAI YAPPIAGNIT CRSNITGLLL TRDGGSNNGV PNDTETFRPG GGDMRNNWRS
ELYKYKVVEV KPLGVAPTEA KRRVVEREKR AVGIGAVFLG ILGAAGSTMG AASITLTVQA
RQLLSGIVQQ QSNLLRAIEA QQHMLQLTVW GIKQLQTRVL AIERYLQDQQ LLGLWGCSGK
LICTTAVPWN TSWSNKSQTD IWDNMTWMQW DKEIGNYTGE IYRLLEESQN QQEKNEKDLL
ALDSWNNLWN WFSISKWLWY IKIFIMIVGG LIGLRIIFAV LSIVNRVRQG Y
SEQ ID NO: 13 (Mos2SEnv DNA)
ATGAGAGTGCGGGGCATGCTGAGAAACTGGCAGCAGTGGTGGATCTGGTCCAGCCTGGGCTTCTGGATGCTGATGATCTACAGCGTGATGG
GCAACCTGTGGGTCACCGTGTACTACGGCGTGCCCGTGTGGAAGGACGCCAAGACCACCCTGTTTTGCGCCTCCGATGCCAAGGCCTACGA
GAAAGAGGTGCACAACGTCTGGGCCACCCACGCCTGTGTGCCCACCGACCCCAATCCCCAGGAAATCGTCCTGGGCAACGTGACCGAGAAC
TTCAACATGTGGAAGAACGACATGGTCGATCAGATGCACGAGGACATCATCTCCCTGTGGGACGCCTCCCTGGAACCCTGCGTGAAGCTGA
CCCCTCTGTGCGTGACCCTGAACTGCCGGAACGTGCGCAACGTGTCCAGCAACGGCACCTACAACATCATCCACAACGAGACATACAAAGA
GATGAAGAACTGCAGCTTCAACGCTACCACCGTGGTCGAGGACCGGAAGCAGAAGGTGCACGCCCTGTTCTACCGGCTGGACATCGTGCCC
CTGGACGAGAACAACAGCAGCGAGAAGTCCTCCGAGAACAGCTCCGAGTACTACAGACTGATCAACTGCAACACCAGCGCCATCACCCAGG
CCTGCCCCAAGGTGTCCTTCGACCCTATCCCCATCCACTACTGCGCCCCTGCCGGCTACGCCATCCTGAAGTGCAACAACAAGACCTTCAA
TGGCACCGGCCCCTGCAACAATGTGTCCACCGTGCAGTGCACCCACGGCATCAAGCCCGTGGTGTCTACCCAGCTGCTGCTGAACGGCAGC
CTGGCCGAGGAAGAGATCATTATCAGAAGCGAGAACCTGACCAACAACGCCAAAACCATCATCGTCCACCTGAACGAAACCGTGAACATCA
CCTGTACCCGGCCTAACAACAACACCCGGAAGTCCATCCGGATCGGCCCTGGCCAGACCTTTTACGCCACCGGCGATATTATCGGCGACAT
CCGGCAGGCCCACTGCAATCTGAGCCGGGACGGCTGGAACAAGACACTGCAGGGCGTCAAGAAGAAGCTGGCCGAACACTTCCCTAACAAG
ACTATCAAGTTCGCCCCTCACTCTGGCGGCGACCTGGAAATCACCACCCACACCTTCAACTGTCGGGGCGAGTTCTTCTACTGCAATACCT
CCAACCTGTTCAACGAGAGCAACATCGAGCGGAACGACAGCATCATCACACTGCCTTGCCGGATCAAGCAGATTATCAATATGTGGCAGGA
AGTGGGCAGAGCCATCTACGCCCCTCCAATCGCCGGCAACATCACATGCCGGTCCAATATCACCGGCCTGCTGCTCACCAGAGATGGCGGC
TCCAACAATGGCGTGCCAAACGACACCGAGACATTCAGACCCGGCGGAGGCGACATGCGGAACAATTGGCGGAGCGAGCTGTACAAGTACA
AGGTGGTGGAAGTGAAGCCCCTGGGCGTGGCCCCTACCGAGGCCAAGAGAAGAGTGGTCGAACGCGAGAAGCGGGCCGTGGGAATCGGAGC
CGTGTTTCTGGGAATCCTGGGAGCCGCTGGCTCTACCATGGGCGCTGCCTCTATCACCCTGACAGTGCAGGCCAGACAGCTGCTCAGCGGC
ATCGTGCAGCAGCAGAGCAACCTGCTGAGAGCCATTGAGGCCCAGCAGCACATGCTGCAGCTGACCGTGTGGGGCATTAAGCAGCTCCAGA
CACGGGTGCTGGCCATCGAGAGATACCTGCAGGATCAGCAGCTCCTGGGCCTGTGGGGCTGTAGCGGCAAGCTGATCTGTACCACCGCCGT
GCCCTGGAATACCTCTTGGAGCAACAAGAGCCAGACCGACATCTGGGACAACATGACCTGGATGCAGTGGGACAAAGAAATCGGCAACTAT
ACCGGCGAGATCTATAGACTGCTGGAAGAGTCCCAGAACCAGCAGGAAAAGAACGAGAAGGACCTGCTGGCCCTGGATTCTTGGAACAATC
TGTGGAACTGGTTCAGCATCTCCAAGTGGCTGTGGTACATCAAGATCTTCATCATGATCGTGGGCGGCCTGATCGGCCTGCGGATCATCTT
TGCCGTGCTGAGCATCGTGAACCGCGTGCGGCAGGGCTAC

### REFERENCES

1. Chen et al. Nat Med. (2001) 7(11), 1225-31

2. UNAIDS Report on the Global AIDS Epidemic 2013

3. Carcelain et al. Immunol Rev. (2013) 254(1), 355-71

4. Katlama Lancet. (2013) 381(9883), 2109-17

5. Gianella et al. Antiviral therapy. (2011) 16(4), 535-45

6. Goujard et al. (2012) Antiviral therapy. 17(6), 1001-9

7. Hamlyn et al. (2012) PLoS one. 7(8), e43754

8. Lodi et al. (2012) Archives of internal medicine. 172(16), 1252-5

9. Saez-Cirion et al. (2013) PLoS pathogens 9(3), e1003211

10. US20120076812

11. Barouch et al., Nat Med 2010, 16:319-323

12. Barouch et al., Cell 155:1-9, 2013

13. Fischer et al, Nat Med, 2007. 13(1): p. 100-6

14. Havenga, et al., 2006, J Gen Virol 87: 2135-43

15. WO 03/104467

16. WO 2007/104792

17. Abbink et al., (2007) Virol 81(9): 4654-63

18. Mayr et al. (1975) Infection 3, 6-14

19. Mayr, A. & Danner, K. (1978), Dev. Biol. Stand. 41: 225-234

20. Stickl (1974), Prey. Med. 3: 97-101

21. Stickl and Hochstein-Mintzel (1971), Munch. Med. Wochenschr. 113: 1149-1153

22. Mayr et al. (1978), Zentralbl. Bacteriol. (B) 167:375-390

23. Blanchard et al. (1998), J. Gen. Virol. 79:1159-116779

24. Carroll & Moss (1997), Virology 238:198-211

25. U.S. Pat. No. 5,185,146; 81

26. WO 02/42480 (US 2003/0206926)

27. WO 03/048184 (US 2006/0159699)

28. U.S. Pat. No. 6,761,893

29. US20110159036

30. U.S. Pat. No. 8,197,825

31. WO 2011/092029

32. Williams et al. (2014) Elife 3, e03821

33. Nkolola et al 2010, J. Virology 84(7): 3270-3279

34. Kovacs et al, PNAS 2012, 109(30):12111-6

35. WO 2010/042942

36. WO 2014/107744

1. A method of inducing an immune response against a human immunodeficiency virus (HIV) in an HIV-infected human subject undergoing antiretroviral therapy (ART), the method comprising administering to the human subject:
(i) a primer vaccine comprising an immunogenically effective amount of one or more adenovirus 26 (Ad26) vectors together encoding four HIV antigens having the amino acid sequences of SEQ ID NO: 1, SEQ ID NO: 12, SEQ ID NO: 3, and SEQ ID NO: 4, and a pharmaceutically acceptable carrier; and
(ii) a booster vaccine comprising an immunogenically effective amount of one or more Modified Vaccinia Ankara (MVA) vectors together encoding four HIV antigens having the amino acid sequences of SEQ ID NO: 1, SEQ ID NO: 3, and SEQ ID NO: 4, and either one of SEQ ID NO: 2 or SEQ ID NO: 12, and a pharmaceutically acceptable carrier.

2. The method of claim 1, wherein the immunogenically effective amount of the one or more Ad26 vectors encoding the four HIV antigens consists of a first Ad26 vector encoding the HIV antigen of SEQ ID NO: 1, a second Ad26 vector encoding the HIV antigen of SEQ ID NO: 12, a third Ad26 vector encoding the HIV antigen of SEQ ID NO: 3, and a fourth Ad26 vector encoding the HIV antigen of SEQ ID NO: 4.

3. The method of claim 1, wherein the immunogenically effective amount of the one or more MVA vectors encoding the four HIV antigens consists of a single MVA vector encoding the HIV antigens of SEQ ID NOs: 1, 3, 4, and either one of SEQ ID NOs: 2 or 12.

4. The method of claim 1, wherein the immunogenically effective amount of the one or more MVA vectors encoding the four HIV antigens consists of a first MVA vector encoding the HIV antigens of SEQ ID NOs: 1 and 3, and a second MVA vector encoding the HIV antigen of SEQ ID NO: 4 and either one of the HIV antigens of SEQ ID NOs: 2 or 12.

5. The method of claim 3, wherein the first, second, third, and fourth Ad26 vectors together are administered at a total dose of about 5×10⁹ to about 1×10¹¹ viral particles (vp) of the Ad26 vectors; and the single MVA vector or the first and second MVA vectors together are administered at a total dose of about 1×10⁷ to about 5×10⁸ plaque-forming units (pfu) of the MVA vector or vectors.

6. The method of claim 1, wherein the booster vaccine is administered at about 22-26 weeks after the primer vaccine is initially administered.

7. The method of claim 1, further comprising re-administering the primer vaccine at about 10-14 weeks after the primer vaccine is initially administered; and re-administering the booster vaccine at about 34 to 38 weeks after the primer vaccine is initially administered.

8. The method of claim 1, further comprising administering to the human subject one or more isolated HIV gp140 envelope polypeptides in combination with the booster vaccine.

9. The method of claim 8, wherein the one or more isolated HIV gp140 envelope polypeptides consists of two trimeric HIV gp140 envelope polypeptides having the amino acid sequences of SEQ ID NO: 9 and SEQ ID NO: 10.

10. The method of claim 1, wherein the human subject has initiated ART outside of the acute phase of HIV infection.

11. The method of claim 1, further comprising administering to the human subject a toll-like receptor 7 (TLR7) agonist.

12. A method of inducing an immune response against a human immunodeficiency virus (HIV) in an HIV-infected human subject undergoing antiretroviral therapy (ART), the method comprising administering to the human subject:
(i) a primer vaccine comprising an immunogenically effective amount of one or more adenovirus 26 (Ad26) vectors together encoding four HIV antigens having the amino acid sequences of SEQ ID NO: 1, SEQ ID NO: 12, SEQ ID NO: 3, and SEQ ID NO: 4, and a pharmaceutically acceptable carrier, in a total dose of about 5×10⁹ to about 1×10″ viral particles (vp) of the Ad26 vectors; and
(ii) a booster vaccine comprising:
(ii,a) a first booster vaccine composition comprising an immunogenically effective amount of one or more adenovirus 26 (Ad26) vectors together encoding four HIV antigens having the amino acid sequences of SEQ ID NO: 1, SEQ ID NO: 12, SEQ ID NO: 3, and SEQ ID NO: 4, and a pharmaceutically acceptable carrier, in a total dose of about 5×10⁹ to about 1×10¹¹ viral particles (vp), vp, of the Ad26 vectors; and
(ii,b) a second booster vaccine composition comprising at least one isolated HIV gp140 envelope polypeptide having an amino acid sequence selected from the group consisting of SEQ ID NO: 9 and SEQ ID NO: 10, an aluminum phosphate adjuvant and a pharmaceutically acceptable carrier, at a total dose of about 125 μg to 350 μg, of the at least one isolated HIV gp140 envelope polypeptide,
wherein the first and second booster vaccine compositions are administered in combination.

13. The method of claim 12, wherein the second booster vaccine composition comprises two trimeric HIV gp140 envelope polypeptides having the amino acid sequences of SEQ ID NO: 9 and SEQ ID NO: 10.

14. The method of claim 12, wherein the human subject has initiated ART outside of the acute phase of HIV infection.

15. The method of claim 12, further comprising administering to the human subject a toll-like receptor 7 (TLR7) agonist.

16. The method of claim 2, wherein the immunogenically effective amount of the one or more MVA vectors encoding the four HIV antigens consists of a single MVA vector encoding the HIV antigens of SEQ ID NOs: 1, 3, 4, and either one of SEQ ID NOs: 2 or 12.

17. The method of claim 2, wherein the immunogenically effective amount of the one or more MVA vectors encoding the four HIV antigens consists of a first MVA vector encoding the HIV antigens of SEQ ID NOs: 1 and 3, and a second MVA vector encoding the HIV antigen of SEQ ID NO: 4 and either one of the HIV antigens of SEQ ID NOs: 2 or 12.

18. The method of claim 5, wherein the first, second, third, and fourth Ad26 vectors together are administered at a total dose of about 5×10¹⁰ vp of the Ad26 vectors; and the single MVA vector or the first and second MVA vectors together are administered at a total dose of about 1×10⁸ pfu of the MVA vector or vectors.

19. The method of claim 12, wherein the total dose of the one or more Ad26 vectors in the primer vaccine is about 5×10¹⁰ vp of the Ad26 vectors; the total dose of the one or more Ad26 vectors in the first booster vaccine composition is about 5×10¹⁰ vp; and the total dose of the at least one isolated HIV gp140 envelope polypeptide in the second booster vaccine composition is about 250 μg.

20. The method of claim 13, wherein the human subject has initiated ART outside of the acute phase of HIV infection.


SEQUENCE LISTING (13 sequences)

SEQ ID NO: 1 - protein, 685 aa, Artificial Sequence
  Mos1.Env protein sequence
MRVTGIRKNYQHLWRWGTMLLGILMICSAAGKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDDVRNVTNNATNTNSSWGEPMEKGEIKNCSFNITTSIRNKVQKQYALFYKLDVVPIDNDSNNTNYRLISCNTSVITQACPKVSFEPIPIHYCAPAGFAILKCNDKKFNGTGPCTNVSTVQCTHGIRPVVSTQLLLNGSLAEEEVVIRSENFTNNAKTIMVQLNVSVEINCTRPNNNTRKSIHIGPGRAFYTAGDIIGDIRQAHCNISRANWNNTLRQIVEKLGKQFGNNKTIVFNHSSGGDPEIVMHSFNCGGEFFYCNSTKLFNSTWTWNNSTWNNTKRSNDTEEHITLPCRIKQIINMWQEVGKAMYAPPIRGQIRCSSNITGLLLTRDGGNDTSGTEIFRPGGGDMRDNWRSELYKYKVVKIEPLGVAPTKAKRRVVQSEKSAVGIGAVFLGFLGAAGSTMGAASMTLTVQARLLLSGIVQQQNNLLRAIEAQQHLLQLTVWGIKQLQARVLAVERYLKDQQLLGIWGCSGKLICTTTVPWNASWSNKSLDKIWNNMTWMEWEREINNYTSLIYTLIEESQNQQEKNEQELLELDKWASLWNWFDISNWLW

SEQ ID NO: 2 - protein, 684 aa, Artificial Sequence
  Mos2.Env protein sequence
MRVRGIQRNWPQWWIWGILGFWMIIICRVMGNLWVTVYYGVPVWKEAKTTLFCASDAKAYEKEVHNVWATHACVPTDPNPQEMVLENVTENFNMWKNDMVDQMHEDIIRLWDQSLKPCVKLTPLCVTLECRNVRNVSSNGTYNIIHNETYKEMKNCSFNATTVVEDRKQKVHALFYRLDIVPLDENNSSEKSSENSSEYYRLINCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNETVNITCTRPNNNTRKSIRIGPGQTFYATGDIIGDIRQAHCNLSRDGWNKTLQGVKKKLAEHFPNKTINFTSSSGGDLEITTHSFNCRGEFFYCNTSGLFNGTYMPNGTNSNSSSNITLPCRIKQIINMWQEVGRAMYAPPIAGNITCRSNITGLLLTRDGGSNNGVPNDTETFRPGGGDMRNNWRSELYKYKVVEVKPLGVAPTEAKRRVVESEKSAVGIGAVFLGILGAAGSTMGAASITLTVQARQLLSGIVQQQSNLLRAIEAQQHMLQLTVWGIKQLQTRVLAIERYLQDQQLLGLWGCSGKLICTTAVPWNTSWSNKSQTDIWDNMTWMQWDKEIGNYTGEIYRLLEESQNQQEKNEKDLLALDSWKNLWNWFDITNWLW

SEQ ID NO: 3 - protein, 1350 aa, Artificial Sequence
  Mos1.Gag-Pol protein sequence
MGARASVLSGGELDRWEKIRLRPGGKKKYRLKHIVWASRELERFAVNPGLLETSEGCRQILGQLQPSLQTGSEELRSLYNTVATLYCVHQRIEIKDTKEALEKIEEEQNKSKKKAQQAAADTGNSSQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPVSILDIRQGPKEPFRDYVDRFYKTLRAEQASQDVKNWMTETLLVQNANPDCKTILKALGPAATLEEMMTACQGVGGPGHKARVLAEAMSQVTNSATIMMQRGNFRNQRKTVKCFNCGKEGHIAKNCRAPRKKGCWKCGKEGHQMKDCTERQANFLGKIWPSNKGRPGNFLQNRPEPTAPPEESFRFGEETTTPSQKQEPIDKEMYPLASLKSLFGNDPSSQMAPISPIETVPVKLKPGMDGPRVKQWPLTEEKIKALTAICEEMEKEGKITKIGPENPYNTPVFAIKKKDSTKWRKLVDFRELNKRTQDFWEVQLGIPHPAGLKKKKSVTVLAVGDAYFSVPLDEGFRKYTAFTIPSTNNETPGIRYQYNVLPQGWKGSPAIFQCSMTRILEPFRAKNPEIVIYQYMAALYVGSDLEIGQHRAKIEELREHLLKWGFTTPDKKHQKEPPFLWMGYELHPDKWTVQPIQLPEKDSWTVNDIQKLVGKLNWASQIYPGIKVRQLCKLLRGAKALTDIVPLTEEAELELAENREILKEPVHGVYYDPSKDLIAEIQKQGHDQWTYQIYQEPFKNLKTGKYAKMRTAHTNDVKQLTEAVQKIAMESIVIWGKTPKFRLPIQKETWETWWTDYWQATWIPEWEFVNTPPLVKLWYQLEKDPIAGVETFYVAGAANRETKLGKAGYVTDRGRQKIVSLTETTNQKTALQAIYLALQDSGSEVNIVTASQYALGIIQAQPDKSESELVNQIIEQLIKKERVYLSWVPAHKGIGGNEQVDKLVSSGIRKVLFLDGIDKAQEEHEKYHSNWRAMASDFNLPPVVAKEIVASCDQCQLKGEAMHGQVDCSPGIWQLACTHLEGKIILVAVHVASGYIEAEVIPAETGQETAYFILKLAGRWPVKVIHTANGSNFTSAAVKAACWWAGIQQEFGIPYNPQSQGVVASMNKELKKIIGQVRDQAEHLKTAVQMAVFIHNFKRKGGIGGYSAGERIIDIIATDIQTKELQKQIIKIQNFRVYYRDSRDPIWKGPAKLLWKGEGAVVIQDNSDIKVVPRRKVKIIKDYGKQMAGADCVAGRQDED

SEQ ID NO: 4 - protein, 1341 aa, Artificial Sequence
  Mos2.Gag-Pol protein sequence
MGARASILRGGKLDKWEKIRLRPGGKKHYMLKHLVWASRELERFALNPGLLETSEGCKQIIKQLQPALQTGTEELRSLFNTVATLYCVHAEIEVRDTKEALDKIEEEQNKSQQKTQQAKEADGKVSQNYPIVQNLQGQMVHQPISPRTLNAWVKVIEEKAFSPEVIPMFTALSEGATPQDLNTMLNTVGGHQAAMQMLKDTINEEAAEWDRLHPVHAGPVAPGQMREPRGSDIAGTTSNLQEQIAWMTSNPPIPVGDIYKRWIILGLNKIVRMYSPTSILDIKQGPKEPFRDYVDRFFKTLRAEQATQDVKNWMTDTLLVQNANPDCKTILRALGPGATLEEMMTACQGVGGPSHKARVLAEAMSQTNSTILMQRSNFKGSKRIVKCFNCGKEGHIARNCRAPRKKGCWKCGKEGHQMKDCTERQANFLGKIWPSHKGRPGNFLQSRPEPTAPPAESFRFEETTPAPKQEPKDREPLTSLRSLFGSDPLSQMAPISPIETVPVKLKPGMDGPKVKQWPLTEEKIKALVEICTEMEKEGKISKIGPENPYNTPIFAIKKKDSTKWRKLVDFRELNKRTQDFWEVQLGIPHPAGLKKKKSVTVLAVGDAYFSVPLDEDFRKYTAFTIPSINNETPGIRYQYNVLPQGWKGSPAIFQSSMTKILEPFRKQNPDIVIYQYMAALYVGSDLEIGQHRTKIEELRQHLLRWGFTTPDKKHQKEPPFLWMGYELHPDKWTVQPIVLPEKDSWTVNDIQKLVGKLNWASQIYAGIKVKQLCKLLRGTKALTEVVPLTEEAELELAENREILKEPVHGVYYDPSKDLIAEIQKQGQGQWTYQIYQEPFKNLKTGKYARMRGAHTNDVKQLTEAVQKIATESIVIWGKTPKFKLPIQKETWEAWWTEYWQATWIPEWEFVNTPPLVKLWYQLEKEPIVGAETFYVAGAANRETKLGKAGYVTDRGRQKVVSLTDTTNQKTALQAIHLALQDSGLEVNIVTASQYALGIIQAQPDKSESELVSQIIEQLIKKEKVYLAWVPAHKGIGGNEQVDKLVSRGIRKVLFLDGIDKAQEEHEKYHSNWRAMASEFNLPPIVAKEIVASCDKCQLKGEAIHGQVDCSPGIWQLACTHLEGKVILVAVHVASGYIEAEVIPAETGQETAYFLLKLAGRWPVKTIHTANGSNFTSATVKAACWWAGIKQEFGIPYNPQSQGVVASINKELKKIIGQVRDQAEHLKTAVQMAVFIHNFKRKGGIGEYSAGERIVDIIASDIQTKELQKQITKIQNFRVYYRDSRDPLWKGPAKLLWKGEGAVVIQDNSDIKVVPRRKAKIIRDYGKQMAGDDCVASRQDED

SEQ ID NO: 5 - DNA, 2055 bp, Artificial Sequence
  Mos1.Env DNA
atgcgggtgaccggcatccggaagaactaccagcacctgtggcggtggggcaccatgctgctgggcatcctgatgatttgctctgccgccggaaagctgtgggtgaccgtgtactacggcgtgcccgtgtggaaagaggccaccaccaccctgttctgcgccagcgacgccaaggcctacgacaccgaggtgcacaacgtgtgggccacccacgcctgcgtgcccaccgaccccaacccccaggaagtggtcctggaaaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcaccgacgacgtgcggaacgtgaccaacaacgccaccaacaccaacagcagctggggcgagcctatggaaaagggcgagatcaagaactgcagcttcaacatcaccacctccatccggaacaaggtgcagaagcagtacgccctgttctacaagctggacgtggtgcccatcgacaacgacagcaacaacaccaactaccggctgatcagctgcaacaccagcgtgatcacccaggcctgccccaaggtgtccttcgagcccatccccatccactactgcgcccctgccggcttcgccatcctgaagtgcaacgacaagaagttcaacggcaccggcccctgcaccaacgtgagcaccgtgcagtgcacccacggcatccggcccgtggtgtccacccagctgctgctgaacggcagcctggccgaggaagaggtggtgatcagaagcgagaatttcaccaacaatgccaagaccatcatggtgcagctgaacgtgagcgtggagatcaactgcacccggcccaacaacaacacccggaagagcatccacatcggccctggcagggccttctacacagccggcgacatcatcggcgacatccggcaggcccactgcaacatcagccgggccaactggaacaacaccctgcggcagatcgtggagaagctgggcaagcagttcggcaacaacaagaccatcgtgttcaaccacagcagcggcggagaccccgagatcgtgatgcacagcttcaactgtggcggcgagttcttctactgcaacagcaccaagctgttcaacagcacctggacctggaacaactccacctggaataacaccaagcggagcaacgacaccgaagagcacatcaccctgccctgccggatcaagcagattatcaatatgtggcaggaggtcggcaaggccatgtacgcccctcccatccggggccagatccggtgcagcagcaacatcaccggcctgctgctgacccgggacggcggcaacgataccagcggcaccgagatcttccggcctggcggcggagatatgcgggacaactggcggagcgagctgtacaagtacaaggtggtgaagatcgagcccctgggcgtggctcccaccaaggccaagcggcgggtggtgcagagcgagaagagcgccgtgggcatcggcgccgtgtttctgggcttcctgggagccgccggaagcaccatgggagccgccagcatgaccctgaccgtgcaggcccggctgctgctgtccggcatcgtgcagcagcagaacaacctgctccgggccatcgaggcccagcagcacctgctgcagctgaccgtgtggggcatcaagcagctgcaggccagggtgctggccgtggagagatacctgaaggatcagcagctcctggggatctggggctgcagcggcaagctgatctgcaccaccaccgtgccctggaacgccagctggtccaacaagagcctggacaagatctggaacaatatgacctggatggaatgggagcgcgagatcaacaattacaccagcctgatctacaccctgatcgaggaaagccagaaccagcaggaaaagaacgagcaggaactgctggaactggacaagtgggccagcctgtggaactggttcgacatcagcaactggctgtgg

SEQ ID NO: 6 - DNA, 2052 bp, Artificial Sequence
  Mos2.Env DNA
atgagagtgcggggcatccagcggaactggccccagtggtggatctggggcatcctgggcttttggatgatcatcatctgccgggtgatgggcaacctgtgggtgaccgtgtactacggcgtgcccgtgtggaaagaggccaagaccaccctgttctgcgccagcgacgccaaggcctacgagaaagaggtgcacaacgtgtgggccacccacgcctgcgtgcccaccgaccccaacccccaggaaatggtcctggaaaacgtgaccgagaacttcaacatgtggaagaacgacatggtggaccagatgcacgaggacatcatccggctgtgggaccagagcctgaagccctgcgtgaagctgacccccctgtgcgtgaccctggaatgccggaacgtgagaaacgtgagcagcaacggcacctacaacatcatccacaacgagacctacaaagagatgaagaactgcagcttcaacgccaccaccgtggtggaggaccggaagcagaaggtgcacgccctgttctaccggctggacatcgtgcccctggacgagaacaacagcagcgagaagtccagcgagaacagctccgagtactaccggctgatcaactgcaacaccagcgccatcacccaggcctgccccaaggtgtccttcgaccccatccccatccactactgcgcccctgccggctacgccatcctgaagtgcaacaacaagaccttcaacggcaccggcccctgcaacaacgtgagcaccgtgcagtgcacccacggcatcaagcccgtggtgtccacccagctgctgctgaacggcagcctggccgaggaagagatcatcatccggtccgagaacctgaccaacaacgccaagaccatcatcgtgcacctgaatgagaccgtgaacatcacctgcacccggcccaacaacaacacccggaagagcatccggatcggccctggccagaccttttacgccaccggcgacatcatcggcgacatccggcaggcccactgcaacctgagccgggacggctggaacaagaccctgcagggcgtgaagaagaagctggccgagcacttccccaataagaccatcaacttcaccagcagcagcggcggagacctggaaatcaccacccacagcttcaactgcaggggcgagttcttctactgcaatacctccggcctgttcaatggcacctacatgcccaacggcaccaacagcaacagcagcagcaacatcaccctgccctgccggatcaagcagatcatcaatatgtggcaggaggtcggcagggccatgtacgcccctcccatcgccggcaatatcacctgccggtccaacatcaccggcctgctgctgaccagggacggcggcagcaacaacggcgtgcctaacgacaccgagaccttccggcctggcggcggagatatgcggaacaactggcggagcgagctgtacaagtacaaggtggtggaggtgaagcccctgggcgtggctcctaccgaggccaagcggcgggtggtggagagcgagaagagcgccgtgggcatcggcgccgtgtttctgggcattctgggagccgccggaagcaccatgggagccgccagcatcaccctgaccgtgcaggcccggcagctgctgtccggcatcgtgcagcagcagagcaacctgctgagagccatcgaggcccagcagcacatgctgcagctgaccgtgtggggcatcaagcagctgcagacccgggtgctggccatcgagagatacctgcaggatcagcagctcctgggcctgtggggctgcagcggcaagctgatctgcaccaccgccgtgccctggaacaccagctggtccaacaagagccagaccgacatctgggacaacatgacctggatgcagtgggacaaagagatcggcaactacaccggcgagatctacaggctgctggaagagagccagaaccagcaggaaaagaacgagaaggacctgctggccctggacagctggaagaacctgtggaactggttcgacatcaccaactggctgtgg

SEQ ID NO: 7 - DNA, 4050 bp, Artificial Sequence
  Mos1.Gag-Pol DNA
atgggagccagagccagcgtgctgtccggaggggagctggaccgctgggagaagatcaggctgaggcctggagggaagaagaagtacaggctgaagcacatcgtgtgggccagcagagagctggaacggtttgccgtgaaccctggcctgctggaaaccagcgagggctgtaggcagattctgggacagctgcagcccagcctgcagacaggcagcgaggaactgcggagcctgtacaacaccgtggccaccctgtactgcgtgcaccagcggatcgagatcaaggacaccaaagaagccctggaaaagatcgaggaagagcagaacaagagcaagaagaaagcccagcaggctgccgctgacacaggcaacagcagccaggtgtcccagaactaccccatcgtgcagaacatccagggacagatggtgcaccaggccatcagccctcggaccctgaacgcctgggtgaaggtggtggaggaaaaggccttcagccctgaggtgatccccatgttctctgccctgagcgagggagccacaccccaggacctgaacaccatgctgaacaccgtgggagggcaccaggctgccatgcagatgctgaaagagacaatcaacgaggaagctgccgagtgggacagggtccacccagtgcacgctggacctatcgctcctggccagatgagagagcccagaggcagcgatattgctggcaccacctccacactgcaggaacagatcggctggatgaccaacaaccctcccatccctgtgggagagatctacaagcggtggatcattctgggactgaacaagatcgtgcggatgtacagccctgtgagcatcctggacatcaggcagggacccaaagagcccttcagggactacgtggaccggttctacaagaccctgagagccgagcaggccagccaggacgtgaagaactggatgaccgagacactgctggtgcagaacgccaaccctgactgcaagaccatcctgaaagccctgggacctgctgccaccctggaagagatgatgacagcctgccagggagtgggaggacctggccacaaggccagggtgctggccgaggccatgagccaggtgaccaactctgccaccatcatgatgcagagaggcaacttccggaaccagagaaagaccgtgaagtgcttcaactgtggcaaagagggacacattgccaagaactgcagggctcccaggaagaaaggctgctggaagtgcggaaaagaaggccaccagatgaaggactgcaccgagaggcaggccaacttcctgggcaagatctggcctagcaacaagggcaggcctggcaacttcctgcagaacagacccgagcccaccgctcctcccgaggaaagcttccggtttggcgaggaaaccaccacccctagccagaagcaggaacccatcgacaaagagatgtaccctctggccagcctgaagagcctgttcggcaacgaccccagcagccagatggctcccatcagcccaatcgagacagtgcctgtgaagctgaagcctggcatggacggacccagggtgaagcagtggcctctgaccgaggaaaagatcaaagccctgacagccatctgcgaggaaatggaaaaagagggcaagatcaccaagatcggacccgagaacccctacaacacccctgtgttcgccatcaagaagaaagacagcaccaagtggaggaaactggtggacttcagagagctgaacaagcggacccaggacttctgggaggtgcagctgggcatccctcaccctgctggcctgaagaaaaagaaaagcgtgaccgtgctggctgtgggagatgcctacttcagcgtgcctctggacgagggcttccggaagtacacagccttcaccatccccagcaccaacaacgagacacctggcatcagataccagtacaacgtgctgcctcagggctggaaaggcagccctgccatcttccagtgcagcatgaccagaatcctggaacccttcagagccaagaaccctgagatcgtgatctaccagtatatggctgccctctacgtgggcagcgacctggaaatcggacagcacagagccaaaatcgaagaactccgcgagcacctgctgaagtggggattcaccacccctgacaagaagcaccagaaagagcctcccttcctgtggatgggctacgagctgcaccctgacaagtggaccgtgcagcccatccagctgccagagaaggactcctggaccgtgaacgacatccagaaactggtcggcaagctgaactgggccagccagatctaccctggcatcaaagtcagacagctgtgtaagctgctgaggggagccaaagcactgaccgacatcgtgcctctgacagaagaagccgagctggaactggccgagaacagagagatcctgaaagaacccgtgcacggagtgtactacgacccctccaaggacctgattgccgagatccagaaacagggacacgaccagtggacctaccagatctatcaggaacctttcaagaacctgaaaacaggcaagtacgccaagatgcggacagcccacaccaacgacgtgaagcagctgaccgaagccgtgcagaaaatcgccatggaaagcatcgtgatctggggaaagacacccaagttcaggctgcccatccagaaagagacatgggaaacctggtggaccgactactggcaggccacctggattcccgagtgggagttcgtgaacaccccacccctggtgaagctgtggtatcagctggaaaaggaccctatcgctggcgtggagacattctacgtggctggagctgccaacagagagacaaagctgggcaaggctggctacgtgaccgacagaggcagacagaaaatcgtgagcctgaccgaaaccaccaaccagaaaacagccctgcaggccatctatctggcactgcaggacagcggaagcgaggtgaacatcgtgacagccagccagtatgccctgggcatcatccaggcccagcctgacaagagcgagagcgagctggtgaaccagatcatcgagcagctgatcaagaaagaacgggtgtacctgagctgggtgccagcccacaagggcatcggagggaacgagcaggtggacaagctggtgtccagcggaatccggaaggtgctgttcctggacggcatcgataaagcccaggaagagcacgagaagtaccacagcaattggagagccatggccagcgacttcaacctgcctcccgtggtggccaaagaaatcgtggccagctgcgaccagtgccagctgaaaggcgaggccatgcacggacaggtggactgctcccctggcatctggcagctggcatgcacccacctggaaggcaagatcattctggtggccgtgcacgtggccagcggatacatcgaagccgaagtgatccctgccgagacagggcaggaaacagcctacttcatcctgaagctggctggcagatggcctgtgaaggtgatccacacagccaacggcagcaacttcacctctgctgccgtgaaggctgcctgttggtgggctggcattcagcaggaatttggcatcccctacaatccccagtctcagggagtggtggccagcatgaacaaagagctgaagaagatcatcggacaggtcagggatcaggccgagcacctgaaaactgccgtccagatggccgtgttcatccacaacttcaagcggaagggagggatcggagggtactctgctggcgagcggatcatcgacatcattgccaccgatatccagaccaaagagctgcagaaacagatcatcaagatccagaacttcagggtgtactacagggacagcagggaccccatctggaagggacctgccaagctgctgtggaaaggcgaaggagccgtcgtcatccaggacaacagcgacatcaaggtggtgcccagacggaaggtgaaaatcatcaaggactacggcaaacagatggctggagccgactgtgtcgctggcaggcaggacgaggac

SEQ ID NO: 8 - DNA, 4023 bp, Artificial Sequence
  Mos2.Gag-Pol DNA
atgggagccagagccagcatcctgcgaggagggaagctggacaagtgggagaagatcaggctgaggcctggagggaagaaacactacatgctgaagcacctggtctgggccagcagagagctggaacggtttgccctcaatcctggcctgctggaaaccagcgagggctgcaagcagatcatcaagcagctgcagcctgccctgcagacaggcaccgaggaactgcggagcctgttcaacaccgtggccaccctgtactgcgtgcatgccgagatcgaagtgagggacaccaaagaagccctggacaagatcgaggaagagcagaacaagagccagcagaaaacccagcaggccaaagaagccgacggcaaggtctcccagaactaccccatcgtgcagaacctgcagggacagatggtgcaccagcccatcagccctcggacactgaatgcctgggtgaaggtgatcgaggaaaaggccttcagccctgaggtgatccccatgttcacagccctgagcgagggagccacaccccaggacctgaacaccatgctgaacaccgtgggagggcaccaggctgccatgcagatgctgaaggacaccatcaacgaggaagctgccgagtgggacaggctgcaccctgtgcacgctggacctgtggctcctggccagatgagagagcccagaggcagcgatattgctggcaccacctccaatctgcaggaacagatcgcctggatgaccagcaaccctcccatccctgtgggagacatctacaagcggtggatcatcctgggactgaacaagatcgtgcggatgtacagccctacctccatcctggacatcaagcagggacccaaagagcctttcagggactacgtggaccggttcttcaagaccctgagagccgagcaggccacccaggacgtgaagaactggatgaccgacaccctgctggtgcagaacgccaaccctgactgcaagaccatcctgagagccctgggacctggagccaccctggaagagatgatgacagcctgccagggagtgggaggaccctctcacaaggctagggtgctggccgaggccatgagccagaccaacagcaccatcctgatgcagcggagcaacttcaagggcagcaagcggatcgtgaagtgcttcaactgtggcaaagagggacacattgccagaaactgtagggcacccaggaagaaaggctgctggaagtgcggaaaagaaggccaccagatgaaggactgcaccgagaggcaggccaacttcctgggcaagatctggcctagccacaagggcagacctggcaacttcctgcagagcagacccgagcccaccgctcctccagccgagagcttccggttcgaggaaaccacccctgctcccaagcaggaacctaaggacagagagcctctgaccagcctgagaagcctgttcggcagcgaccctctgagccagatggctcccatctcccctatcgagacagtgcctgtgaagctgaagcctggcatggacggacccaaggtgaaacagtggcctctgaccgaggaaaagatcaaagccctggtggagatctgtaccgagatggaaaaagagggcaagatcagcaagatcggacccgagaacccctacaacacccctatcttcgccatcaagaagaaagacagcaccaagtggaggaaactggtggacttcagagagctgaacaagcggacccaggacttctgggaggtgcagctgggcatccctcaccctgctggcctgaagaaaaagaaaagcgtgaccgtgctggccgtgggagatgcctacttcagcgtgcctctggacgaggacttcagaaagtacacagccttcaccatccccagcatcaacaacgagacacctggcatcagataccagtacaacgtgctgcctcagggatggaagggctctcctgcaatcttccagagcagcatgaccaagatcctggaacccttccggaagcagaaccctgacatcgtgatctaccagtacatggcagccctgtacgtcggcagcgacctggaaatcggacagcaccggaccaagatcgaagaactcaggcagcacctgctgcggtggggattcaccacccctgacaagaagcaccagaaagagcctcccttcctgtggatgggctacgagctgcacccagacaagtggaccgtgcagcccatcgtgctgcctgagaaggactcctggaccgtgaacgacatccagaaactggtcggcaagctgaactgggccagccagatctacgctggcatcaaagtgaagcagctgtgtaagctcctgagaggcaccaaagccctgaccgaggtggtgccactgacagaggaagccgagctggaactggccgagaacagagagatcctgaaagaacccgtgcacggagtgtactacgaccccagcaaggacctgattgccgagatccagaagcagggacagggacagtggacctaccagatctaccaggaacccttcaagaacctgaaaacaggcaagtacgccaggatgaggggagcccacaccaacgacgtcaaacagctgaccgaagccgtgcagaagatcgccaccgagagcatcgtgatttggggaaagacacccaagttcaagctgcccatccagaaagagacatgggaggcctggtggaccgagtactggcaggccacctggattcccgagtgggagttcgtgaacaccccacccctggtgaagctgtggtatcagctggaaaaagaacccatcgtgggagccgagacattctacgtggctggagctgccaacagagagacaaagctgggcaaggctggctacgtgaccgacagaggcaggcagaaagtggtgtccctgaccgataccaccaaccagaaaacagccctgcaggccatccacctggctctgcaggactctggcctggaagtgaacatcgtgacagccagccagtatgccctgggcatcattcaggcacagcctgacaagagcgagagcgagctggtgtctcagatcattgagcagctgatcaagaaagaaaaggtgtacctggcctgggtgccagcccacaaggggatcggagggaacgagcaggtggacaagctggtgtccaggggcatccggaaggtgctgtttctggacggcatcgacaaagcccaggaagagcacgagaagtaccacagcaattggagagccatggccagcgagttcaacctgcctcccatcgtggccaaagaaatcgtggcctcttgcgacaagtgccagctgaaaggcgaggccattcacggacaggtggactgcagcccaggcatctggcagctggcctgcacccacctggaaggcaaggtgatcctggtggccgtgcacgtggcctctggatacatcgaagccgaagtgatccctgccgagacaggccaggaaacagcctacttcctgctgaagctggctggcaggtggcctgtgaaaaccatccacacagccaacggcagcaacttcacctctgccaccgtgaaggctgcctgttggtgggctggcattaagcaggaatttggcatcccctacaaccctcagtctcagggagtggtggcctccatcaacaaagagctgaagaagatcatcggacaggtcagggatcaggccgagcatctgaaaacagccgtccagatggccgtgttcatccacaacttcaagcggaagggagggatcggagagtactctgctggcgagaggatcgtggacattatcgccagcgatatccagaccaaagaactgcagaagcagatcacaaagatccagaacttcagggtgtactacagggacagcagagatcccctgtggaagggacctgccaagctgctgtggaaaggcgaaggagccgtcgtcatccaggacaacagcgacatcaaggtggtgcccagacggaaggccaagatcatcagagactacggcaaacagatggctggcgacgactgcgtcgcctctaggcaggacgaggac

SEQ ID NO: 9 - protein, 679 aa, Artificial Sequence
  Clade C gp140 protein
AENLWVGNMWVTVYYGVPVWTDAKTTLFCASDTKAYDREVHNVWATHACVPTDPNPQEIVLENVTENFNMWKNDMVDQMHEDIISLWDQSLKPCVKLTPLCVTLHCTNATFKNNVTNDMNKEIRNCSFNTTTEIRDKKQQGYALFYRPDIVLLKENRNNSNNSEYILINCNASTITQACPKVNFDPIPIHYCAPAGYAILKCNNKTFSGKGPCNNVSTVQCTHGIKPVVSTQLLLNGSLAEKEIIIRSENLTDNVKTIIVHLNKSVEIVCTRPNNNTRKSMRIGPGQTFYATGDIIGDIRQAYCNISGSKWNETLKRVKEKLQENYNNNKTIKFAPSSGGDLEITTHSFNCRGEFFYCNTTRLFNNNATEDETITLPCRIKQIINMWQGVGRAMYAPPIAGNITCKSNITGLLLVRDGGEDNKTEEIFRPGGGNMKDNWRSELYKYKVIELKPLGIAPTGAKERVVEREERAVGIGAVFLGFLGAAGSTMGAASLTLTVQARQLLSSIVQQQSNLLRAIEAQQHMLQLTVWGIKQLQTRVLAIERYLKDQQLLGIWGCSGKLICTTNVPWNSSWSNKSQTDIWNNMTWMEWDREISNYTDTIYRLLEDSQTQQEKNEKDLLALDSWKNLWSWFDISNWLWYIKSRIEGRGSGGYIPEAPRDGQAYVRKDGEWVLLSTFL

SEQ ID NO: 10 - protein, 695 aa, Artificial Sequence
  Mosaic gp140 protein
AGKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDDVRNVTNNATNTNSSWGEPMEKGEIKNCSFNITTSIRNKVQKQYALFYKLDVVPIDNDSNNTNYRLISCNTSVITQACPKVSFEPIPIHYCAPAGFAILKCNDKKFNGTGPCTNVSTVQCTHGIRPVVSTQLLLNGSLAEEEVVIRSENFTNNAKTIMVQLNVSVEINCTRPNNNTRKSIHIGPGRAFYTAGDIIGDIRQAHCNISRANWNNTLRQIVEKLGKQFGNNKTIVFNHSSGGDPEIVMHSFNCGGEFFYCNSTKLFNSTWTWNNSTWNNTKRSNDTEEHITLPCRIKQIINMWQEVGKAMYAPPIRGQIRCSSNITGLLLTRDGGNDTSGTEIFRPGGGDMRDNWRSELYKYKVVKIEPLGVAPTKAKERVVQREERAVGIGAVFLGFLGAAGSTMGAASMTLTVQARLLLSGIVQQQNNLLRAIEAQQHLLQLTVWGIKQLQARVLAVERYLKDQQLLGIWGCSGKLICTTTVPWNASWSNKSLDKIWNNMTWMEWEREINNYTSLIYTLIEESQNQQEKNEQELLELDKWASLWNWFDISNWLWYIKSRIEGRGSGGYIPEAPRDGQAYVRKDGEWVLLSTFL

SEQ ID NO: 11 - protein, 29 aa, Artificial Sequence
  exemplary leader sequence for gp140 stabilized trimeric protein production
MRVRGIQRNCQHLWRWGTLILGMLMICSA

SEQ ID NO: 12 - protein, 711 aa, Artificial Sequence
  Mos2S.Env protein sequence
MRVRGMLRNWQQWWIWSSLGFWMLMIYSVMGNLWVTVYYGVPVWKDAKTTLFCASDAKAYEKEVHNVWATHACVPTDPNPQEIVLGNVTENFNMWKNDMVDQMHEDIISLWDASLEPCVKLTPLCVTLNCRNVRNVSSNGTYNIIHNETYKEMKNCSFNATTVVEDRKQKVHALFYRLDIVPLDENNSSEKSSENSSEYYRLINCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNETVNITCTRPNNNTRKSIRIGPGQTFYATGDIIGDIRQAHCNLSRDGWNKTLQGVKKKLAEHFPNKTIKFAPHSGGDLEITTHTFNCRGEFFYCNTSNLFNESNIERNDSIITLPCRIKQIINMWQEVGRAIYAPPIAGNITCRSNITGLLLTRDGGSNNGVPNDTETFRPGGGDMRNNWRSELYKYKVVEVKPLGVAPTEAKRRVVEREKRAVGIGAVFLGILGAAGSTMGAASITLTVQARQLLSGIVQQQSNLLRAIEAQQHMLQLTVWGIKQLQTRVLAIERYLQDQQLLGLWGCSGKLICTTAVPWNTSWSNKSQTDIWDNMTWMQWDKEIGNYTGEIYRLLEESQNQQEKNEKDLLALDSWNNLWNWFSISKWLWYIKIFIMIVGGLIGLRIIFAVLSIVNRVRQGY

SEQ ID NO: 13 - DNA, 2133 bp, Artificial Sequence
  Mos2S.Env DNA
atgagagtgcggggcatgctgagaaactggcagcagtggtggatctggtccagcctgggcttctggatgctgatgatctacagcgtgatgggcaacctgtgggtcaccgtgtactacggcgtgcccgtgtggaaggacgccaagaccaccctgttttgcgcctccgatgccaaggcctacgagaaagaggtgcacaacgtctgggccacccacgcctgtgtgcccaccgaccccaatccccaggaaatcgtcctgggcaacgtgaccgagaacttcaacatgtggaagaacgacatggtcgatcagatgcacgaggacatcatctccctgtgggacgcctccctggaaccctgcgtgaagctgacccctctgtgcgtgaccctgaactgccggaacgtgcgcaacgtgtccagcaacggcacctacaacatcatccacaacgagacatacaaagagatgaagaactgcagcttcaacgctaccaccgtggtcgaggaccggaagcagaaggtgcacgccctgttctaccggctggacatcgtgcccctggacgagaacaacagcagcgagaagtcctccgagaacagctccgagtactacagactgatcaactgcaacaccagcgccatcacccaggcctgccccaaggtgtccttcgaccctatccccatccactactgcgcccctgccggctacgccatcctgaagtgcaacaacaagaccttcaatggcaccggcccctgcaacaatgtgtccaccgtgcagtgcacccacggcatcaagcccgtggtgtctacccagctgctgctgaacggcagcctggccgaggaagagatcattatcagaagcgagaacctgaccaacaacgccaaaaccatcatcgtccacctgaacgaaaccgtgaacatcacctgtacccggcctaacaacaacacccggaagtccatccggatcggccctggccagaccttttacgccaccggcgatattatcggcgacatccggcaggcccactgcaatctgagccgggacggctggaacaagacactgcagggcgtcaagaagaagctggccgaacacttccctaacaagactatcaagttcgcccctcactctggcggcgacctggaaatcaccacccacaccttcaactgtcggggcgagttcttctactgcaatacctccaacctgttcaacgagagcaacatcgagcggaacgacagcatcatcacactgccttgccggatcaagcagattatcaatatgtggcaggaagtgggcagagccatctacgcccctccaatcgccggcaacatcacatgccggtccaatatcaccggcctgctgctcaccagagatggcggctccaacaatggcgtgccaaacgacaccgagacattcagacccggcggaggcgacatgcggaacaattggcggagcgagctgtacaagtacaaggtggtggaagtgaagcccctgggcgtggcccctaccgaggccaagagaagagtggtcgaacgcgagaagcgggccgtgggaatcggagccgtgtttctgggaatcctgggagccgctggctctaccatgggcgctgcctctatcaccctgacagtgcaggccagacagctgctcagcggcatcgtgcagcagcagagcaacctgctgagagccattgaggcccagcagcacatgctgcagctgaccgtgtggggcattaagcagctccagacacgggtgctggccatcgagagatacctgcaggatcagcagctcctgggcctgtggggctgtagcggcaagctgatctgtaccaccgccgtgccctggaatacctcttggagcaacaagagccagaccgacatctgggacaacatgacctggatgcagtgggacaaagaaatcggcaactataccggcgagatctatagactgctggaagagtcccagaaccagcaggaaaagaacgagaaggacctgctggccctggattcttggaacaatctgtggaactggttcagcatctccaagtggctgtggtacatcaagatcttcatcatgatcgtgggcggcctgatcggcctgcggatcatctttgccgtgctgagcatcgtgaaccgcgtgcggcagggctac